(19) United States
(12) Patent Application Publication
(10) Pub. No.: US 2001/0001144 A1
(43) Pub. Date: May 10, 2001

(54) Pharmacy drug management system providing patient specific drug dosing, drug interaction analysis, order generation, and patient data matching

(76) Inventor: Thomas L. Kapp, Katy, TX (US)

Correspondence Address: AKIN, GUMP, STRAUSS, HAUER & FELD, 711 LOUISIANA STREET, SUITE 1900 SOUTH, HOUSTON, TX, 77002, US

(21) Appl. No.: 09/748,594

(22) Filed: Dec. 22, 2000

Related U.S. Application Data
(63) Continuation of application No. 09/032,512, filed on Feb. 27, 1998, now abandoned.

(51) Int. Cl. G06F 17/60; A61M 37/00

(52) U.S. Cl. 705/3; 604/131

(57) ABSTRACT

A pharmacy drug management system provides pharmacy drug management software for patient-specific drug dosing, drug interaction analysis, order generation, and patient data matching. When a drug is added for a patient, the system detects if the drug is a doser drug requiring precise therapeutic dosing and also detects if the drug will cause any drug interaction problems for the patient, reducing the likelihood of clinical misjudgments. The system checks for drug interaction problems resulting from drugs, food allergies, and the medical condition of the patient. An on-screen order may then be generated. A doctor or pharmacist thus is aware of any drug interaction problems before writing an order for the patient. If a selected drug is a doser drug, the system uses phannacokinetic equations specific to the patient data to calculate the appropriate therapeutic dosing parameters. Through a therapy management module of the pharmacy drug management software, a clinical professional may access a formulary listing available drugs, advisories for drugs, drug and medical condition information help files, an infusion calculator, a note for recording patient events, access to a patient data matching database for locating therapies for patients with similar medical conditions to the particular patient, and other therapy tools, all from the screen of a computer system running the software.

### RELATED APPLICATIONS

1. This application is a continuation of U.S. application Ser. No. 09/032,512, filed Feb. 27, 1998, incorporated herein in its entirety for reference.

### BACKGROUND OF THE INVENTION

2. 1. Field of the Invention

3. The present invention relates to a comprehensive pharmacy drug management system for preventing iatrogenic drug effects, and more particularly to a pharmacy drug management system providing a single pharmacy drug management software package for patient specific drug dosing, drug interaction analysis, order generation, and patient data matching.

4. 2. Description of the Related Art

5. Iatrogenic illnesses (illnesses caused by the medical profession) have been a significant cause of disease and death of patients. Most iatrogenic illnesses result from complications of drug therapy. Adverse drug reactions have been the cause of roughly 10% of all hospital admissions. Thirty six percent or more of hospitalized patients have their problems compounded by suffering iatrogenic drug effects. We could assume then that many ambulatory patients, especially those on numerous medications and suffering a variety of ailments, are also candidates for iatrogenic drug problems. Further, it is believed that iatrogenic drug illnesses cost the American economy many billions of dollars a year.

6. National statistics from the insurance industry estimate that 28% of all medical malpractice suits are the results of improper use of medications. It is widely thought that, medical malpractice suits for adverse drug reactions will increase five fold over the next few years as lawyers and patients become more sophisticated as to their understanding of iatrogenic drug problems and their complexities. In many cases where there are no errors in clinical procedure or judgment, many will try to distort the relevant facts. The latter scenarios are predicated on the assumptions that physicians will not specifically address the issue, continue to practice as before, and hope that all potential problems never materialize.

7. Within a hospital, numerous orders for drugs causing adverse drug reactions for patients are written a day. Preparing and processing an order begins with a doctor physically writing an order. The order is then entered by a nurse into a computer connected to a pharmacy database so that the order may be processed. While the order is being processed, the doctor depending on the time of day is busy with other patients or has left the hospital. The order may then come up on the screen of the computer indicating there is a drug interaction problem. The ordered drug may have a problem interacting with another drug prescribed for the patient. The ordered drug might also negatively impact the patient's medical condition.

8. Drug interaction information for certain drugs is stored in the pharmacy database. If either type of problem is detected by the computer system, then a message pops up on the screen of the computer system indicating a drug interaction problem. The doctor is then called or paged and requested to prepare a new order. Meanwhile, the patient who is in need of immediate drug therapy must wait for the doctor to write a new order. If the drug selected for the new order also causes a drug interaction problem detected by the computer system, then filling the order is again delayed. The conventional system for preparing and processing an order thus not only creates an order without taking patient-specific data into account (particularly since an order is physically written) but also checks for drug interaction problems after an order has already been written.

9. Adverse drug reactions are particularly significant in geriatric pharmacology. Elderly persons often have multiple chronic diseases and are under multiple medications, increasing concern regarding drug-drug (or drug to drug) and drug-disease interactions. Many common symptoms of the elderly (e.g., gastrointestinal problems, dizziness, and mental status changes) can be difficult to distinguish from drug side effects or may be caused and exacerbated by medications. Introduction of a new medication into the regime of an elderly individual is thus fraught with adverse possibilities.

10. Overdosing and underdosing of drugs has also contributed to numerous iatrogenic illnesses. For certain classes of drugs such as aminoglycosides and cephalosporins, precise therapeutic dosing levels must be determined. The goal of the medical profession has been to avoid overdosing and underdosing by tailoring drug administration to an individual patient's needs. In pursuit of this goal, the medical profession has predominately utilized pharmacokinetic principles in drug dosing. The basic pharmacokinetic parameters, which include volume of distribution, rate of metabolizing, rate of excretion, rate of absorption and half-life, are commonly used in equations for calculating dosing amounts and the dosing integral for drugs requiring precise therapeutic dosing levels. However, so far as is known, the medical profession has lacked a capability of automatically identifying a drug needing precise therapeutic dosing and then quickly utilizing pharmacokinetic principles and patient-specific data to dose a patient for the drug.

11. The administration of drug therapy has required clinical professionals to use numerous distinct and dispersed tools and resources, such as a formulary listing available drugs, an infusion calculator, a pharmacy database, patient records, clinical reports, and drug-specific advisories. For the medical profession, some inconvenience is necessarily suffered due to reliance upon these different tools which are often not readily accessible. The time a clinical professional needs to determine drug therapy for a patient is a significant factor for patients in need of immediate therapy. The significant time required by clinical professionals to locate and consult various resources has thus prolonged the waiting period for patients.

### SUMMARY OF THE INVENTION

12. Briefly, the present invention provides a pharmacy drug management system for monitoring and correcting iatrogenic drug illnesses so as to deliver optimum drug therapy to a patient in a managed care environment. The pharmacy drug management system provides patient specific drug dosing, drug interaction analysis, order generation, and patient data matching. The modules provided by the pharmacy drug management software include a drug interaction analysis sub-module, a drug dosing module, an order generation module, and a patient data matching module. Through the drug interaction analysis sub-module, each drug to be prescribed will be examined for potential problems associated with other drugs and medical data of the patient such as the medical condition, allergy and food of the patient. The module allows the input of detailed medical history, allergies, diet and prescribed drugs from all physicians being seen by the patient, drugs that are intended to be prescribed, and any non-prescription medications that are being used. The module then checks for drug to drug interactions and drug interactions based on the medical condition of the patient. In this way, the module will alert the physician and clinical pharmacist of the potential drug interaction problems before they occur. The module also provides advisories concerning particular drugs and recommendations of alternate drugs to use in place of certain drugs. The detection and correction of drug interaction problems by the drug interaction analysis sub-module serve to minimize clinical liability for adverse drug reactions. Clinical liability for adverse drug reactions is further managed through tracking the adverse drug reaction efficiency for each doctor and pharmacist. This is the ultimate conceptual system of managed care practice of preventive medicine vis-a-vis prescribing.

13. The drug interaction analysis sub-module is contained in a therapy management module supporting a variety of features. These features include a note or internal chart for maintaining a continuous chain of events for a patient, an infusion calculator for computing an infusion rate for a drug, a worksheet for listing the current drugs of the patient, advisories for providing information specific to particular drugs and information specific to particular classes of patients, a list of the current medical conditions of a patient, a formulary listing the available drugs, a list of the drugs of the patient resulting in a “drug interaction” warning, and a list of drugs of a patient resulting in a contradiction warning. The doctor or pharmacist thus is provided with an integrated interface for using numerous drug therapy tools simultaneously.

14. The drug dosing module determines precise therapeutic drug dosing levels for drugs having a narrow therapeutic index. Such drugs, for example, may include aminoglycosides, cephlosporins, antibiotics, cardiovascular disease drugs, and pulmonary disease drugs. The module uses patient specific data and pharmacokinetic principles to properly dose the patient. The module also provides dosing guidelines based on a programmed clinical judgment in response to particular modifying factors (factors influencing creatinine clearance) of the patient. The module serves as a therapeutic monitor by predicting the levels of a drug within a patient and providing review of a therapeutic range for the patient. When the drug dosing module is used in combination with the drug interaction analysis sub-module, the pharmacy drug management system becomes an online clinical consultant. Through the use of the drug interaction analysis sub-module and the drug dosing module, the patient's course of therapy is set.

15. After the patient's course of therapy is set by the drug dosing module and the drug interaction analysis sub-module, using the order generation module, a doctor or pharmacist processes an on-screen order. The on-screen order includes the standard components of a written order. In addition to the order, the screen provides a hospital formulary so that specific drugs provided by a drug manufacturer may be selected for entry into the order. The order is printed out as a label that is affixed to a container for the drug and is also printed out as a prescription for the patient. The order module also includes the capability to recreate an order for generation of a renewal order, to control drug inventory, and to reorder drugs when a drug reaches a predetermined amount.

16. The patient data matching module calls a patient data matching database external to the computer system to match the current patient's medical condition to previous patient therapies that have been administered for treatment of patients with similar medical conditions. Through the entry of specific data, the module will extract similar patient parameters meeting the criteria of the medical condition. The clinical professional can then review the matched patients, complete with the drug therapy administered for treatment and the clinical outcomes. The clinician can continue the course that has been set for the patient or alter the course of therapy based on the clinical data from the matches. In the disclosed embodiment, the patient data matching database is a relational database provided on a website.

### BRIEF DESCRIPTION OF THE DRAWINGS

17. A better understanding of the present invention can be obtained when the following detailed description of the preferred embodiment is considered in conjunction with the following drawings, in which:

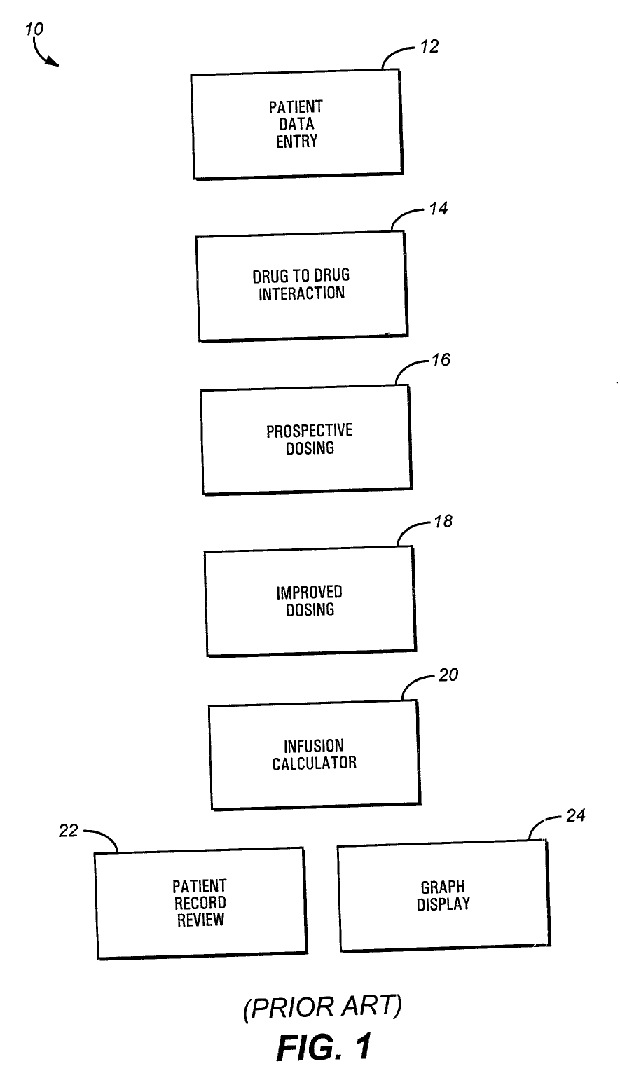

18. FIG. 1 is a block diagram of drug management software in accordance with prior art invented by Applicant;

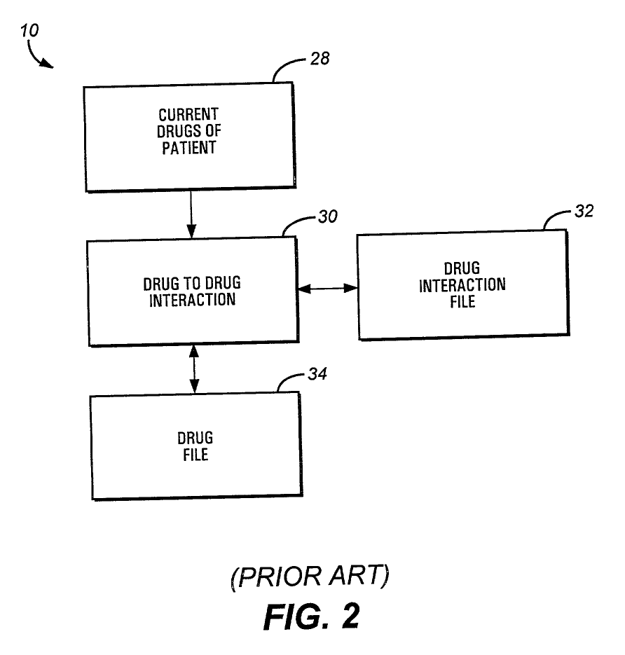

19. FIG. 2 is a schematic block diagram of the relationship between a drug to drug interaction module and associated data blocks of the drug management software of FIG. 1;

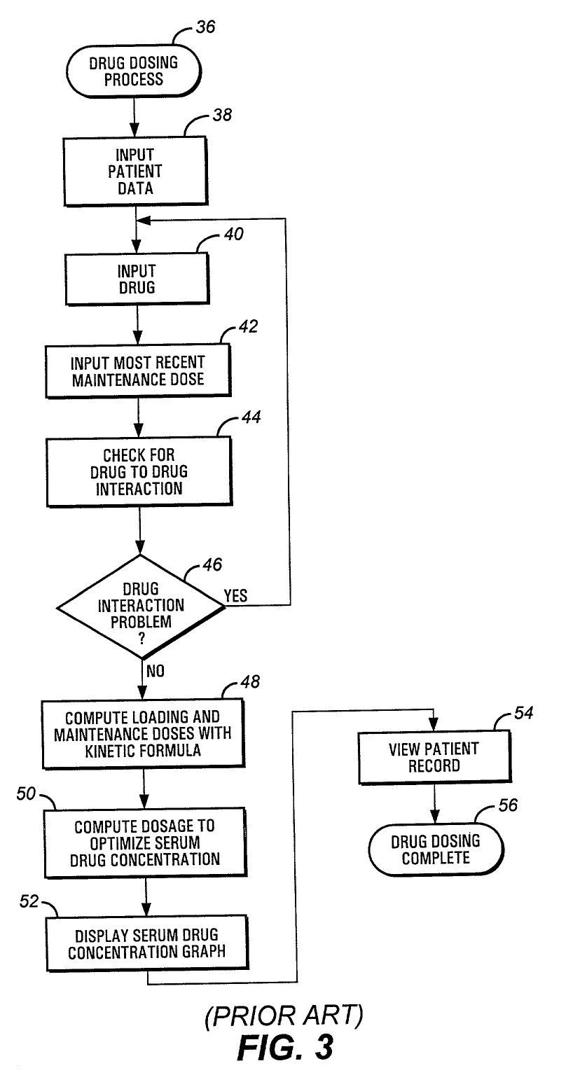

20. FIG. 3 is a flow chart of a drug dosing process performed by the drug management software of FIG. 1;

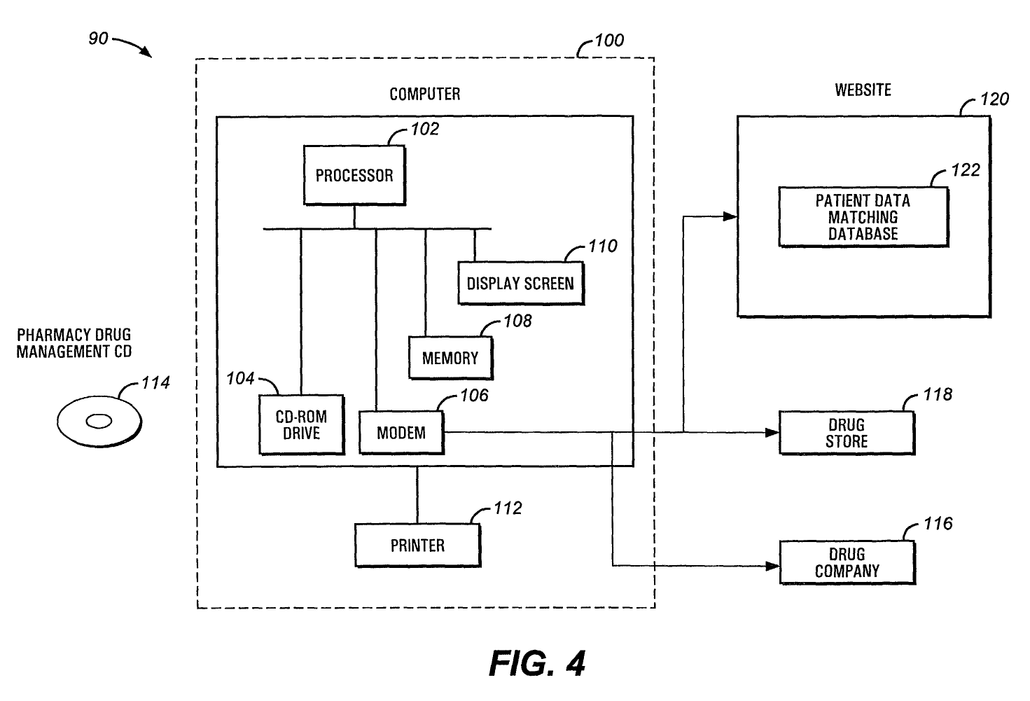

21. FIG. 4 is a schematic diagram of a pharmacy drug management system including pharmacy drug management software in accordance with the present invention;

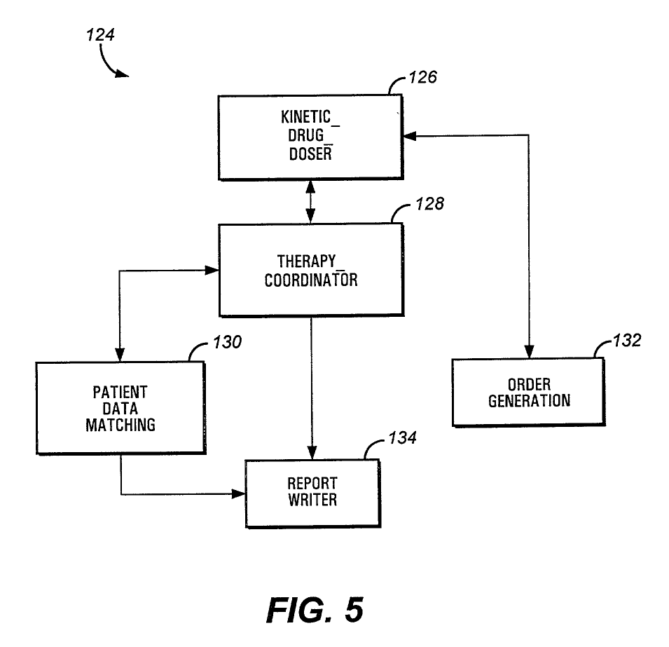

22. FIG. 5 is a schematic block diagram of modules of the pharmacy drug management software of FIG. 4;

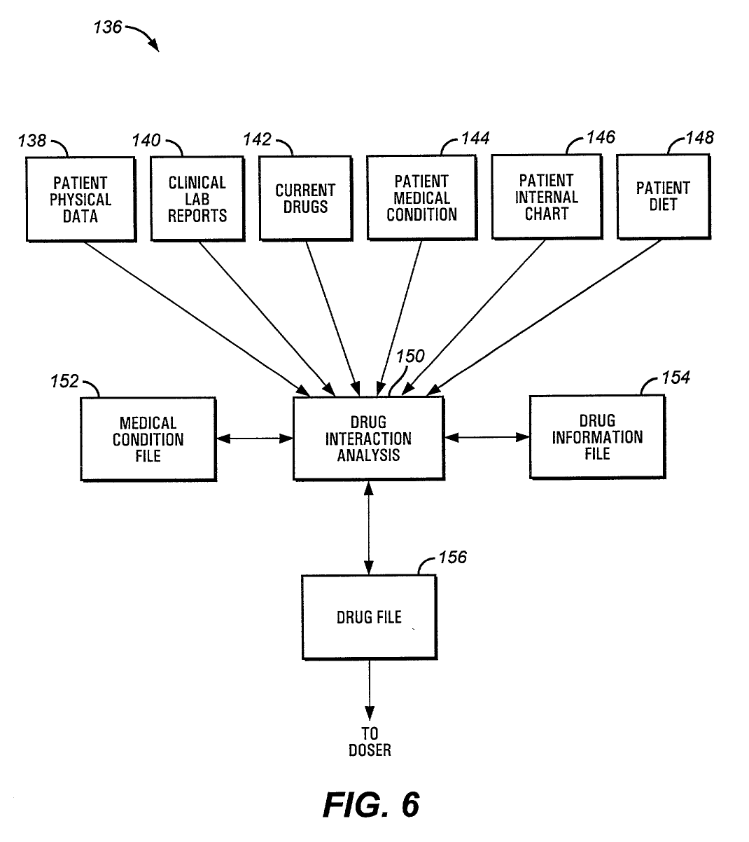

23. FIG. 6 is a schematic block diagram of the relationship between the drug interaction analysis sub-module of the THERAPY_COORDINATOR module of FIG. 5 and associated data blocks;

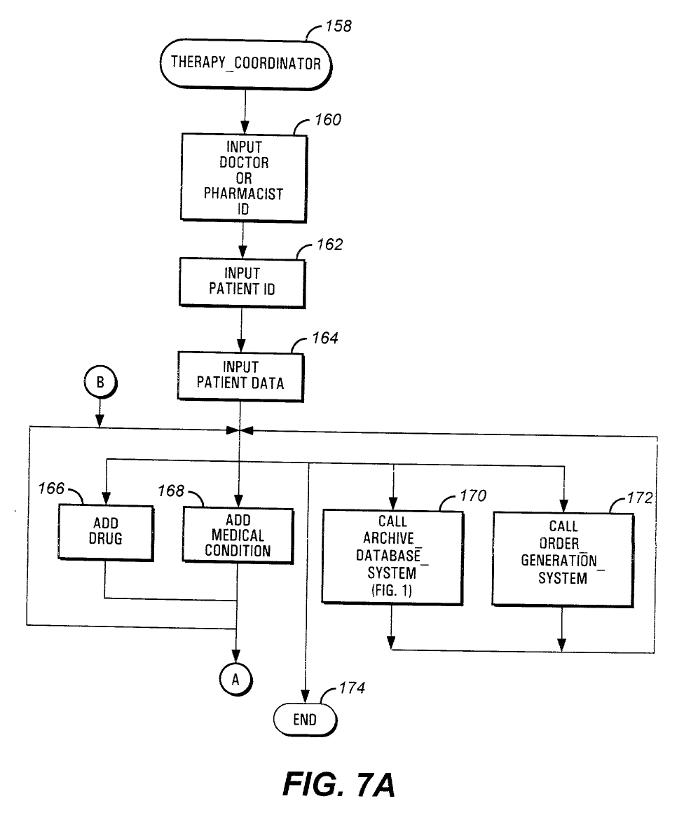

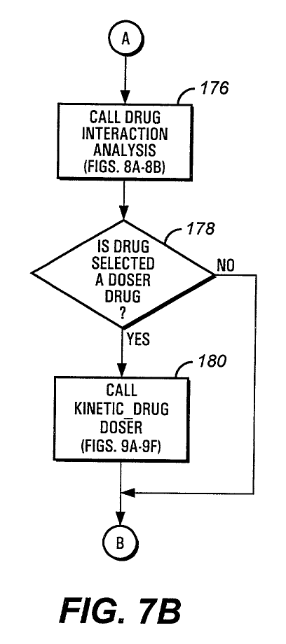

24. FIGS. 7A-7B are flow charts of a drug management process performed by the computer system of FIG. 4 in executing the pharmacy drug management software of FIG. 4;

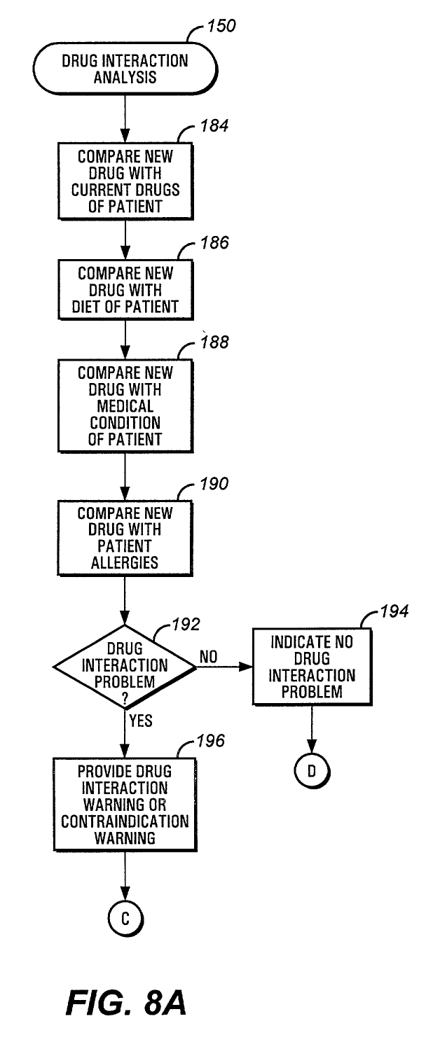

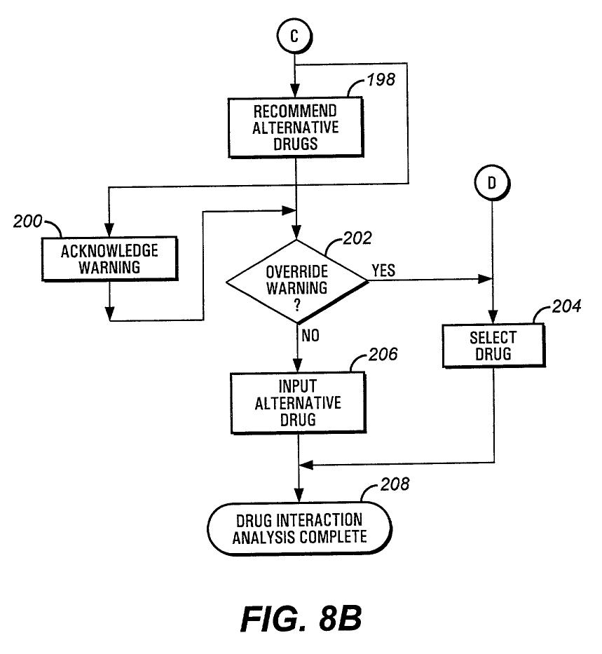

25. FIGS. 8A-8B are flow charts of the THERAPY_COORDINATOR module of FIG. 5 called by the drug management process of FIGS. 7A-7B;

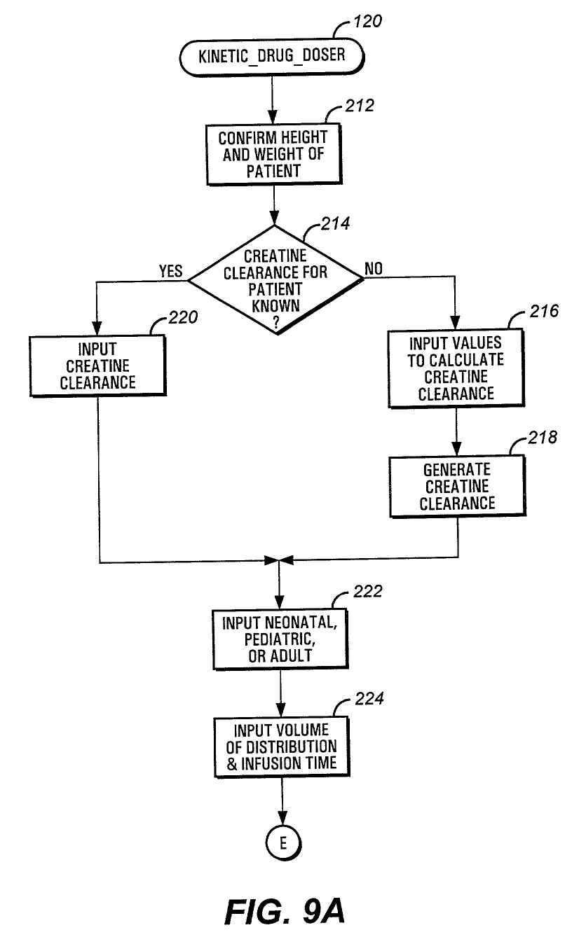

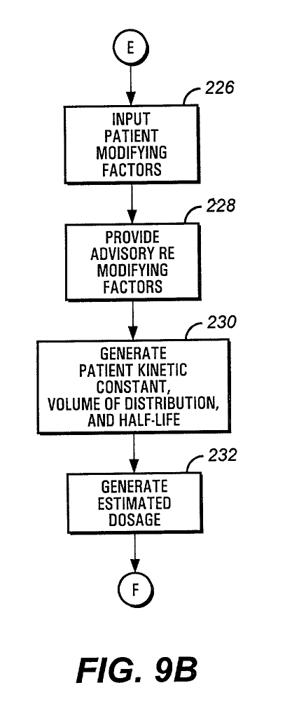

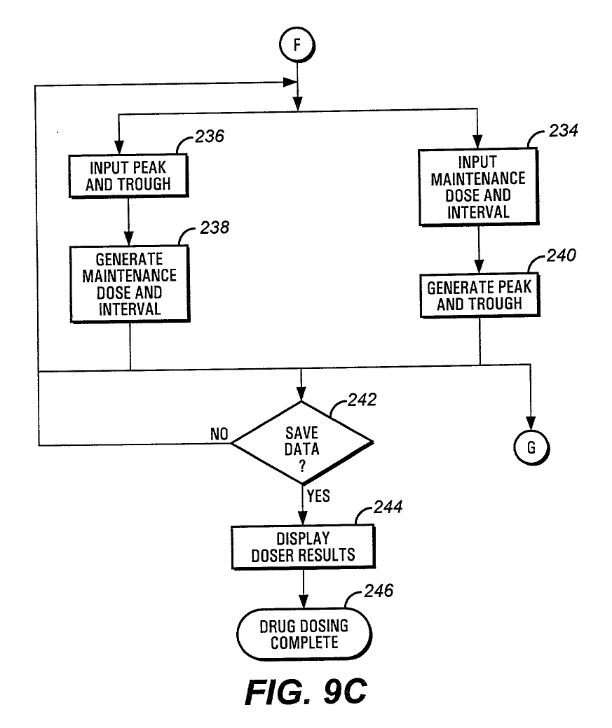

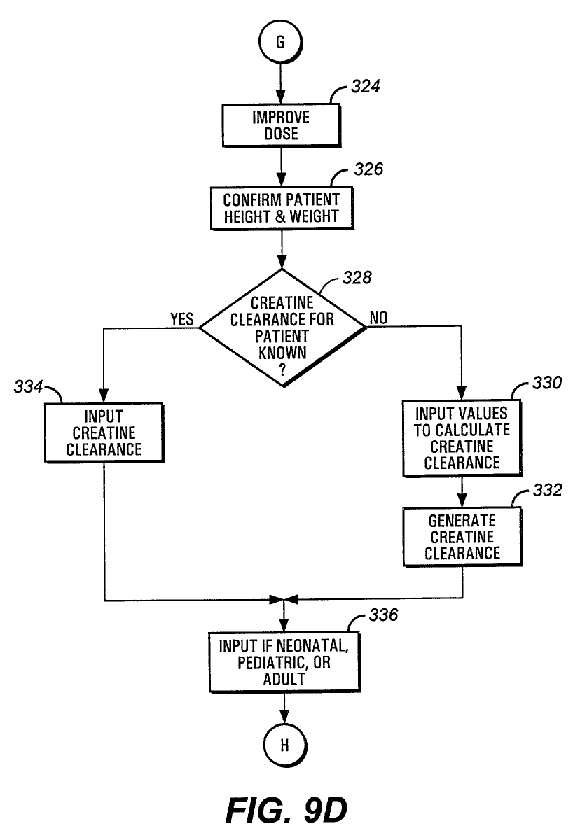

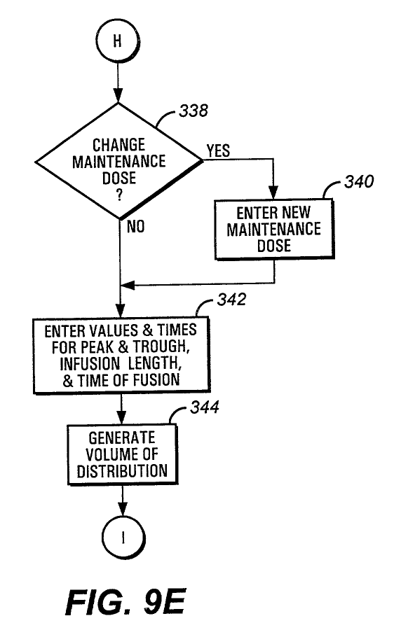

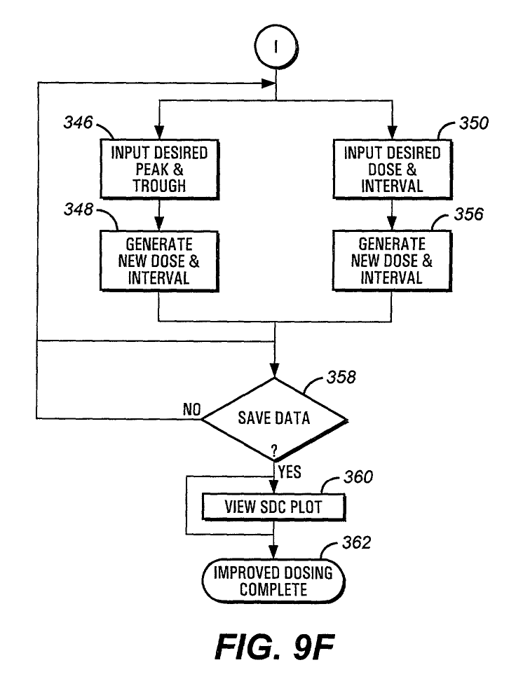

26. FIGS. 9A-9F are flow charts of the KINETIC_DRUG_DOSER module called by the drug management process of FIGS. 7A-7B;

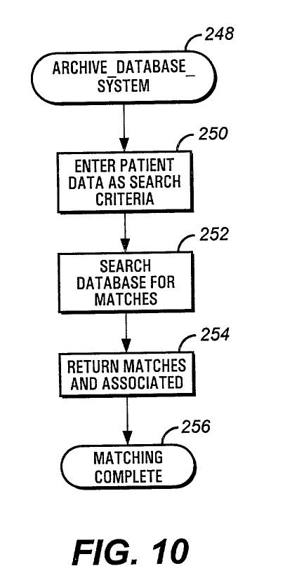

27. FIG. 10 is a flow chart of an ARCHIVE_DATABASE_SYSTEM module called by the drug management process of FIGS. 7A-7B;

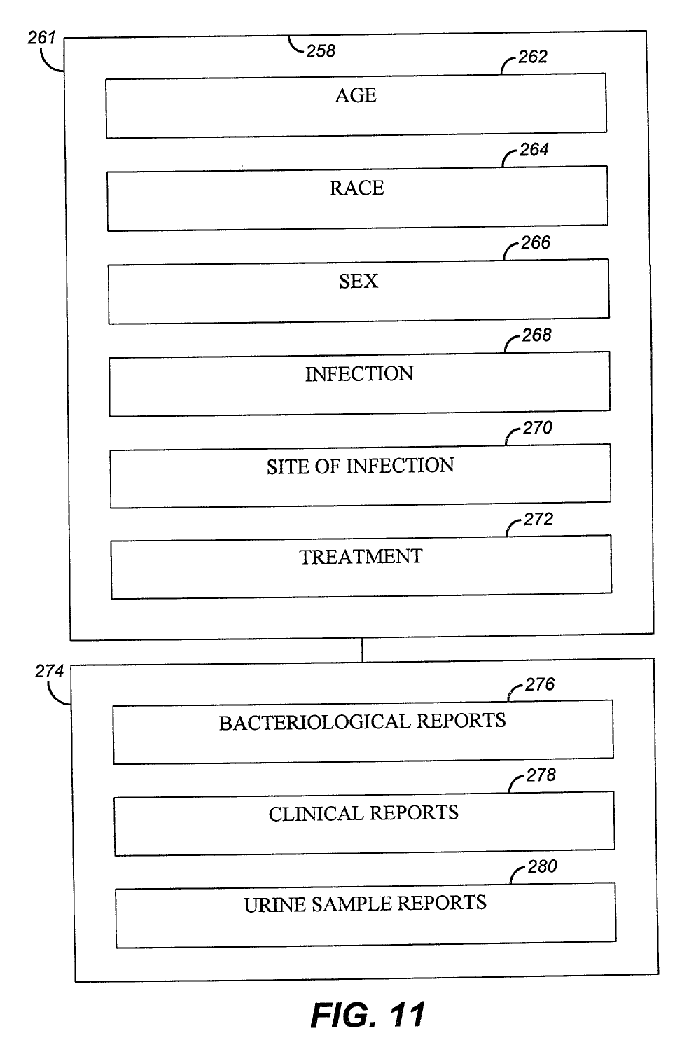

28. FIG. 11 is an illustration of fields of an exemplary record for storing patient data in the patient data module matching database of FIG. 4;

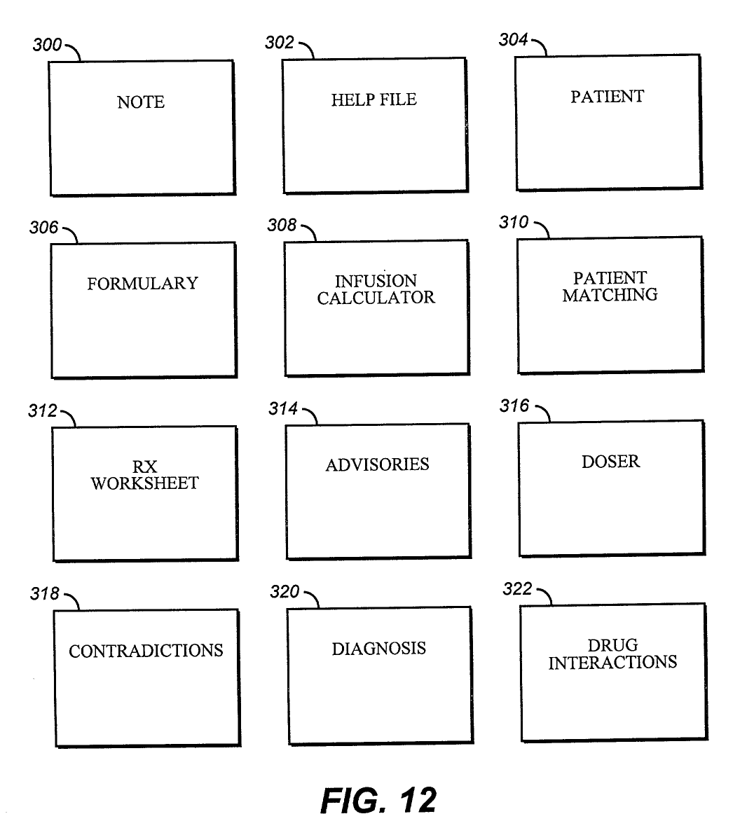

29. FIG. 12 is a block diagram of features of the pharmacy drug management software of FIG. 4 accessible from the THERAPY_COORDINATOR module of FIG. 5;

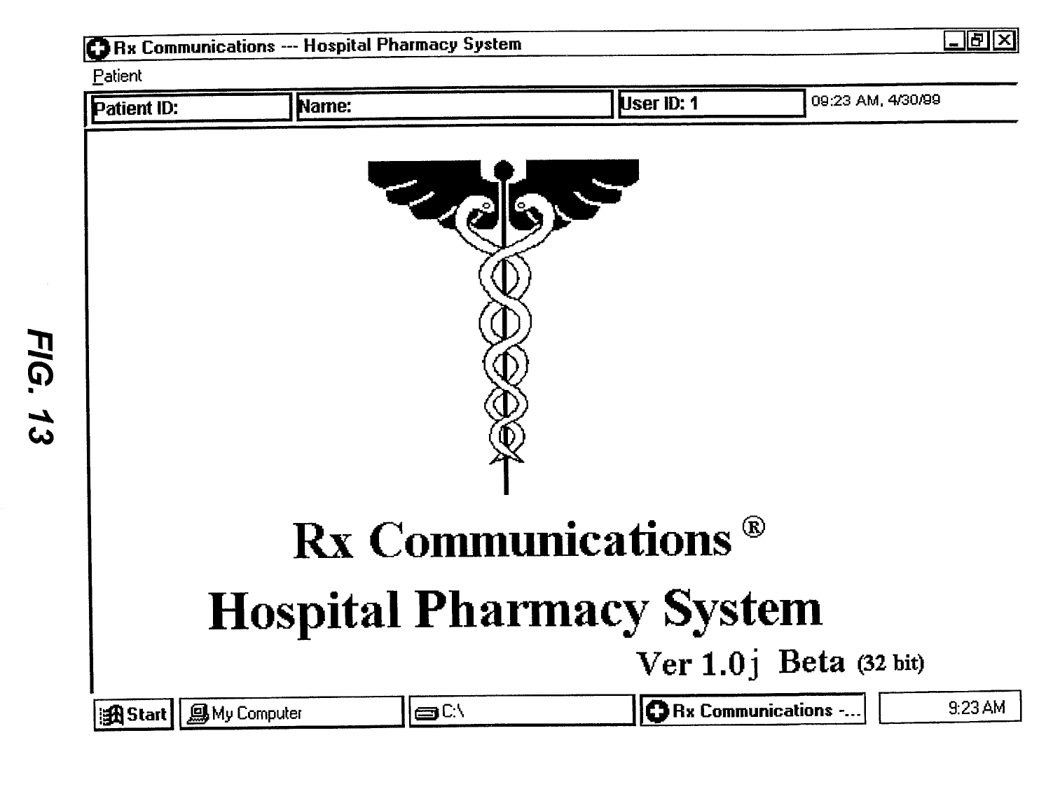

30. FIG. 13 is an illustration of an exemplary opening window displayed by the pharmacy drug management software of FIG. 4;

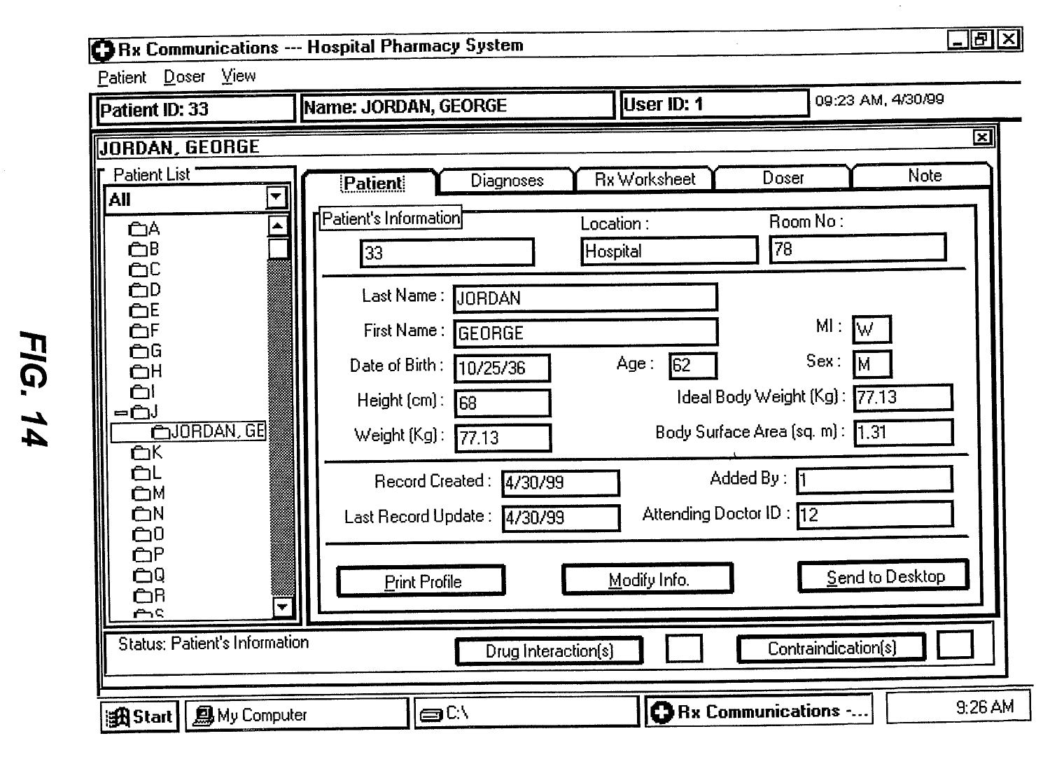

31. FIG. 14 is an illustration of an exemplary patient data window displayed by the pharmacy drug management software of FIG. 4;

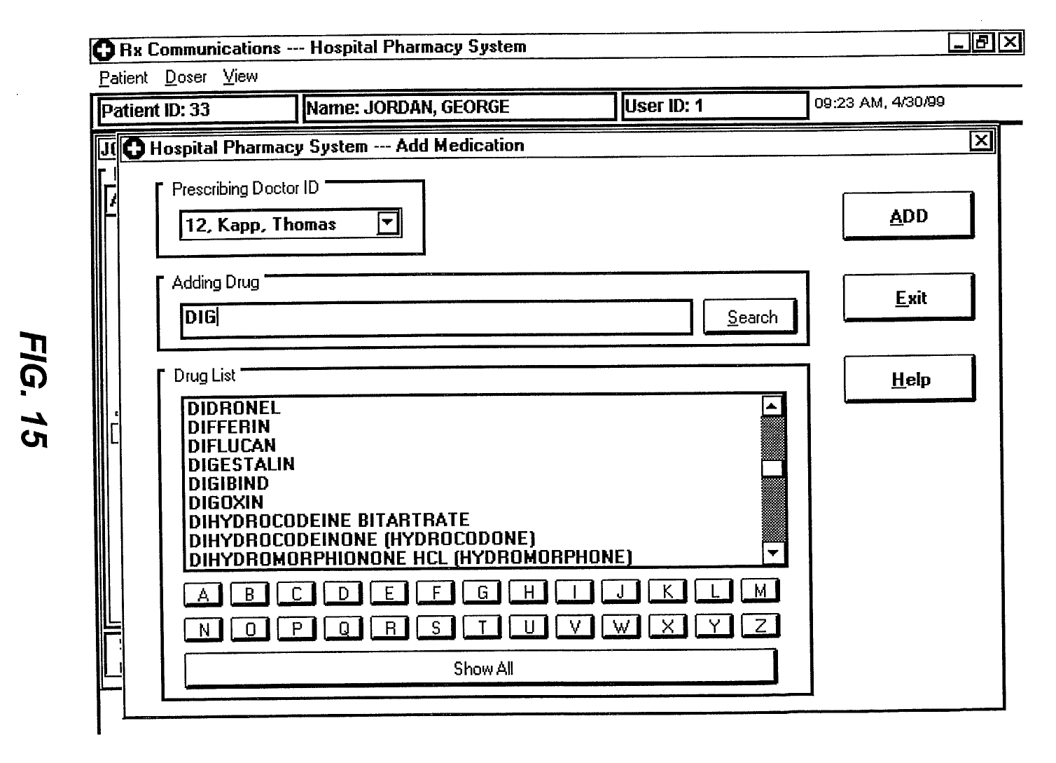

32. FIG. 15 is an illustration of an exemplary add medication window displayed by the pharmacy drug management software of FIG. 4;

33. FIG. 16 is an illustration of an exemplary drug interaction warning window displayed by the THERAPY_COORDINATOR module of FIG. 5;

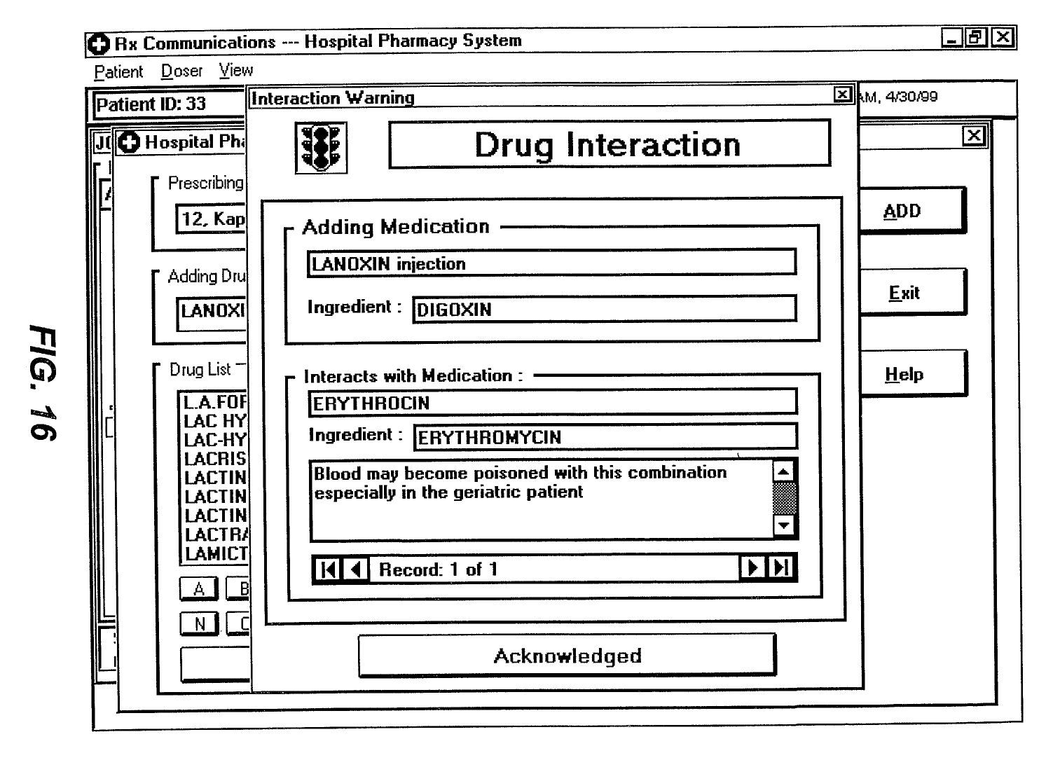

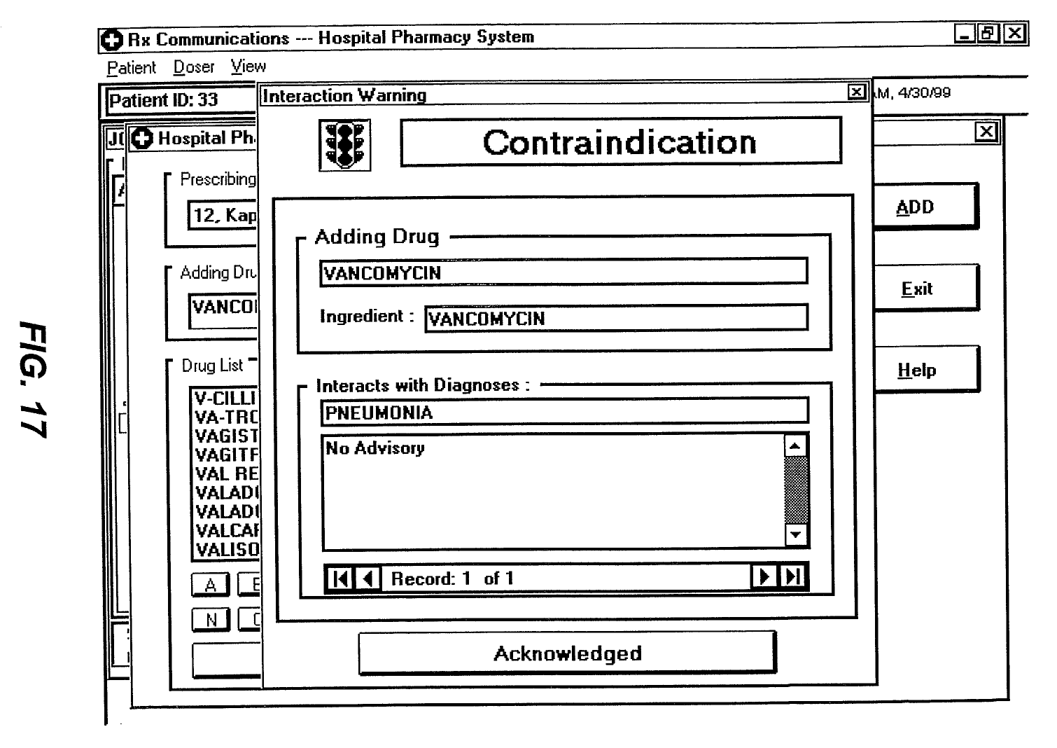

34. FIG. 17 is an illustration of an exemplary contraindication warning window displayed by the THERAPY COORDINATOR module of FIG. 5;

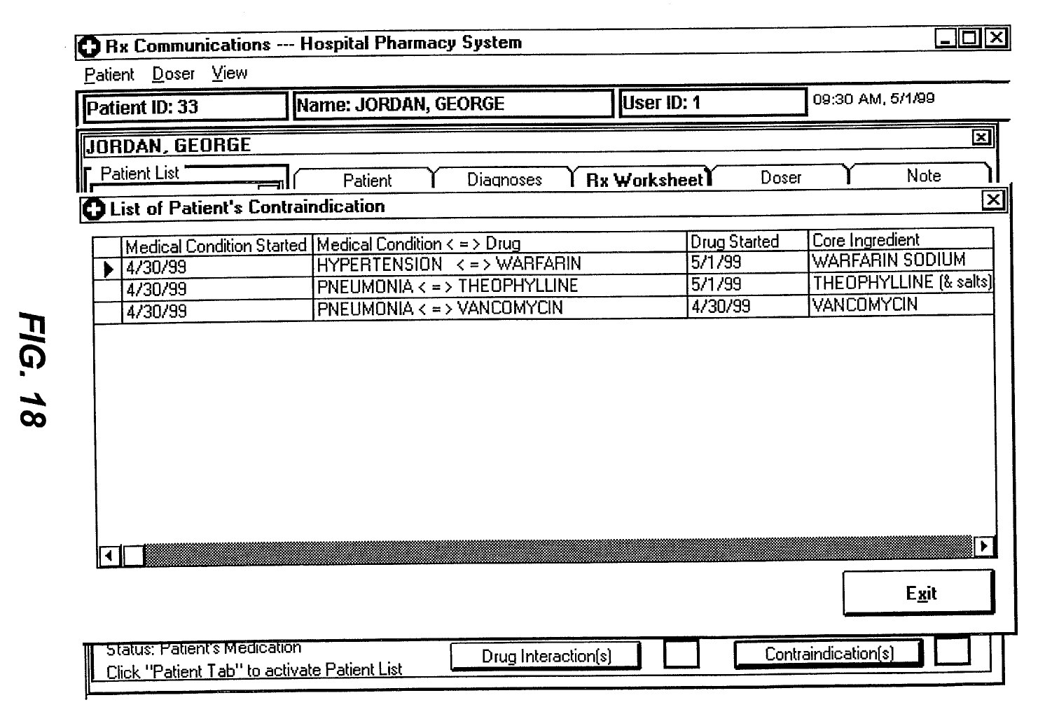

35. FIG. 18 is an illustration of an exemplary Rx Worksheet window depicting a contraindications list by the THERAPY_COORDINATOR module of FIG. 5;

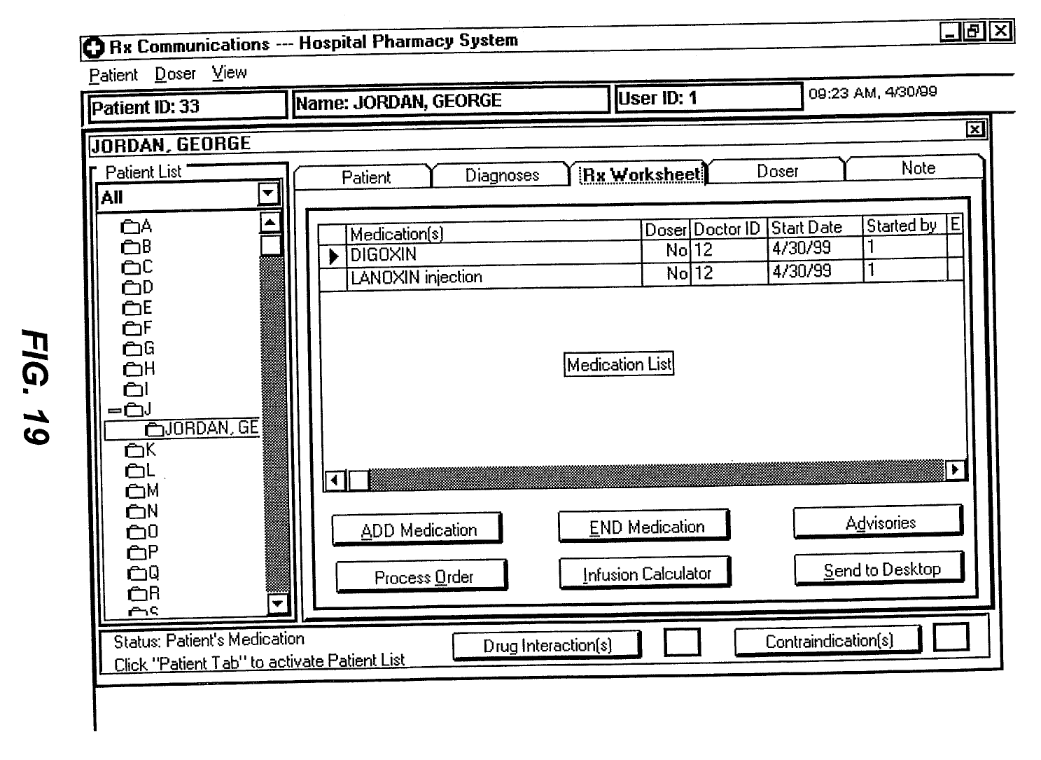

36. FIG. 19 is an illustration of an exemplary Rx Worksheet window depicting a medication list by the THERAPY_COORDINATOR module of FIG. 5;

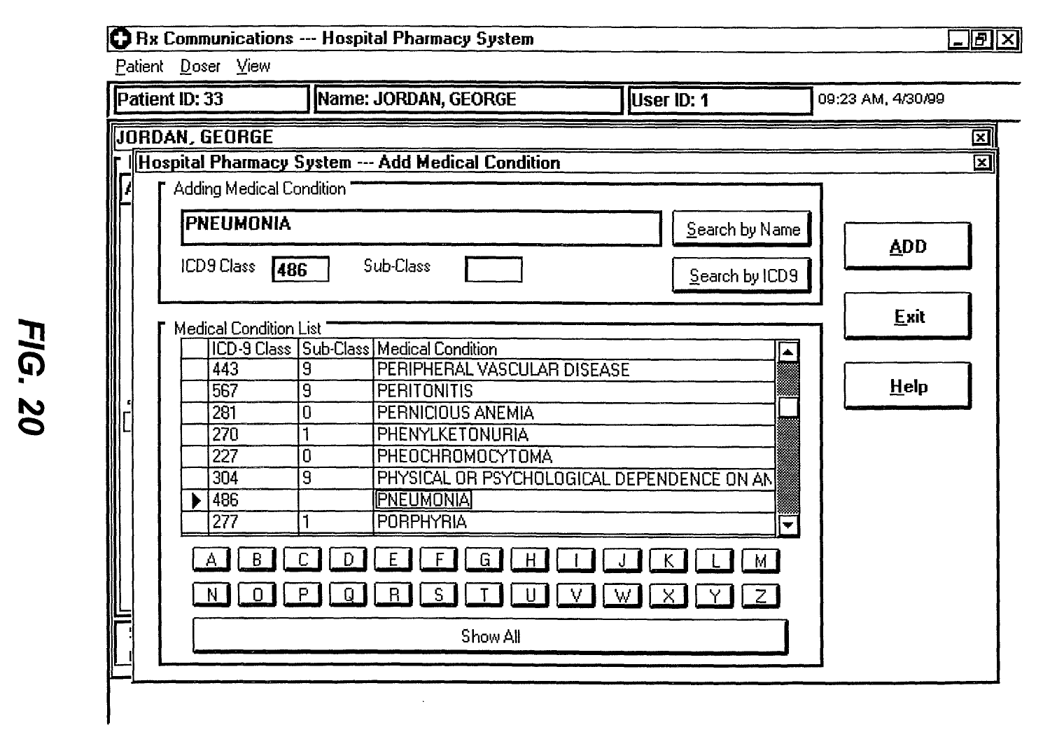

37. FIG. 20 is an illustration of an exemplary add medical condition window displayed by the THERAPY_COORDINATOR module of FIG. 5;

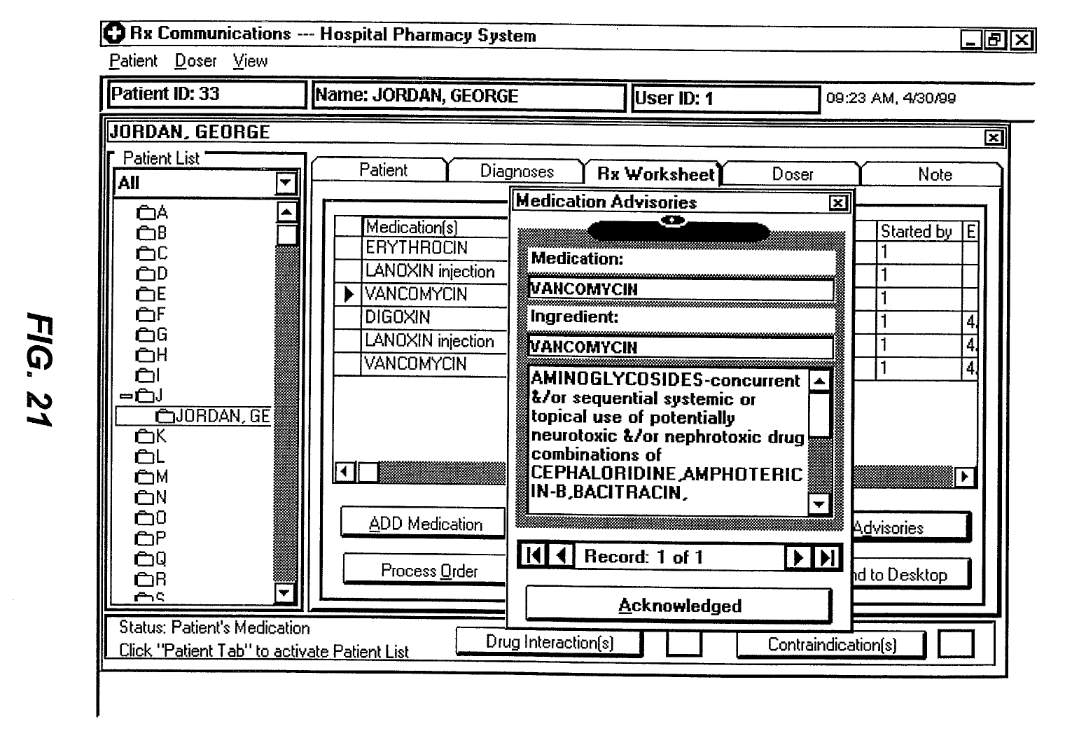

38. FIG. 21 is an illustration of an exemplary medication advisory window displayed by the THERAPY_COORDINATOR module of FIG. 5;

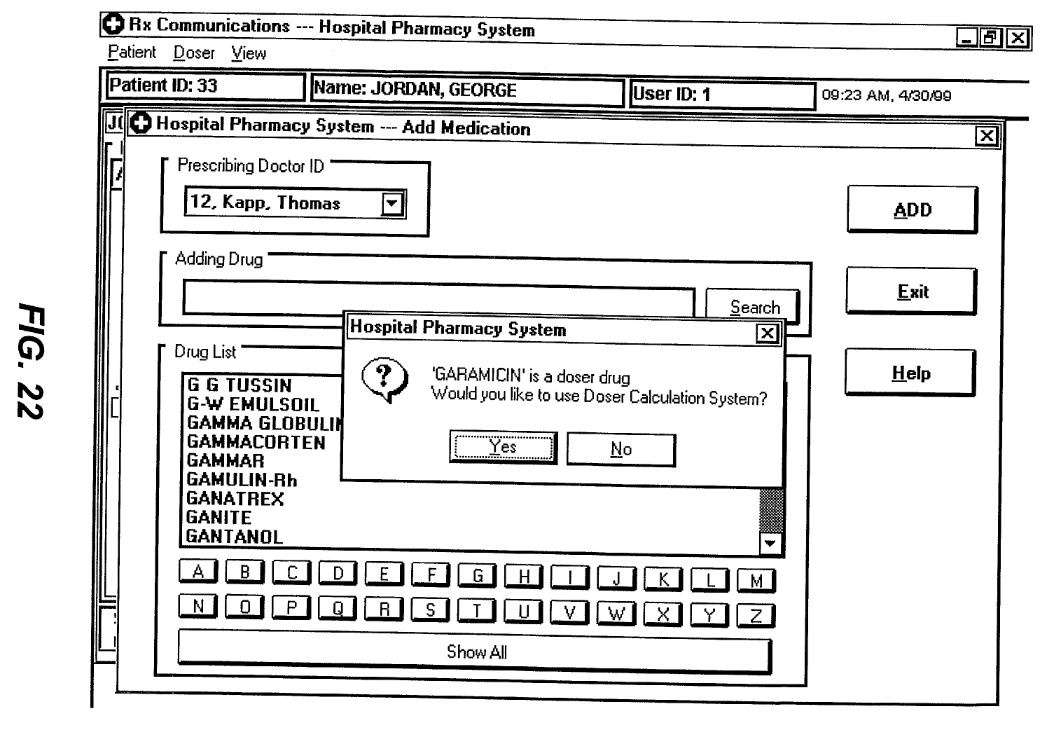

39. FIG. 22 is an illustration of an exemplary doser drug indication window displayed by the THERAPY_COORDINATOR module of FIG. 5;

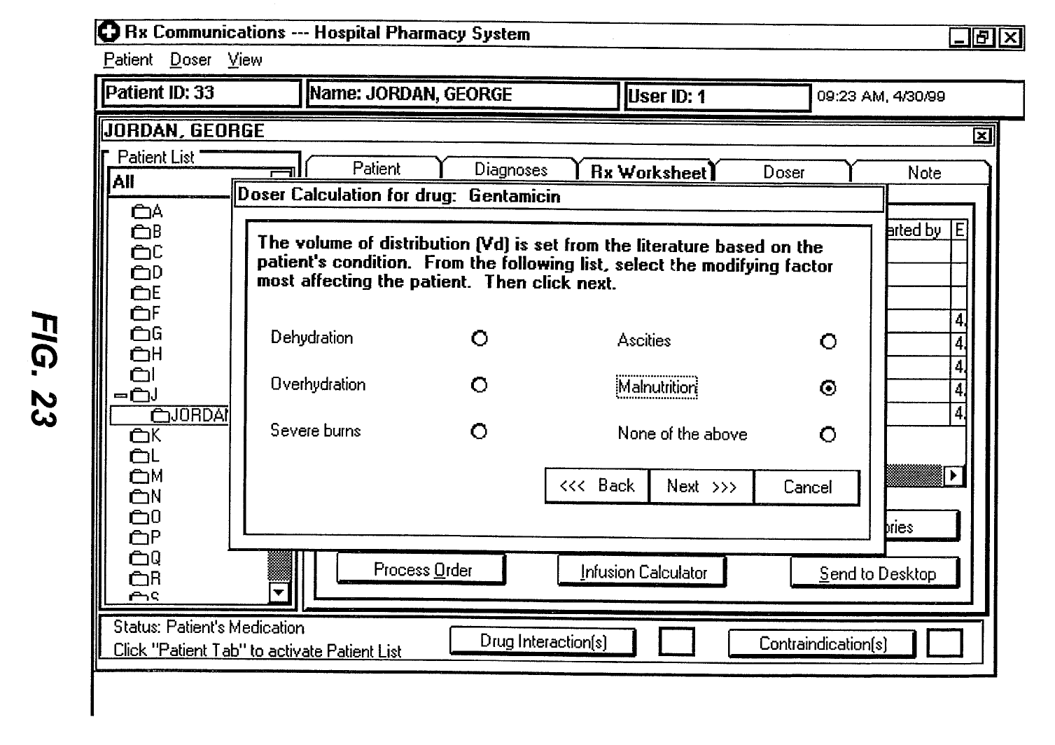

40. FIG. 23 is an illustration of an exemplary modifying factors window displayed by the KINETIC_DRUG_DOSER module of FIGS. 9A-9F;

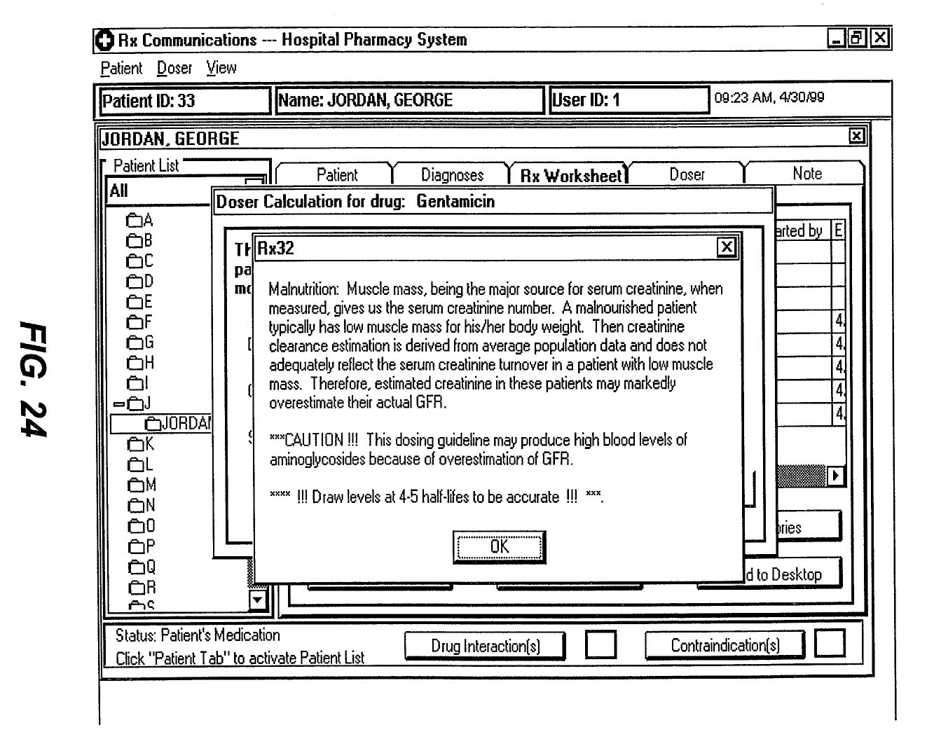

41. FIG. 24 is an illustration of an exemplary malnutrition window displayed by the KINETIC_DRUG_DOSER module of FIGS. 9A-9F;

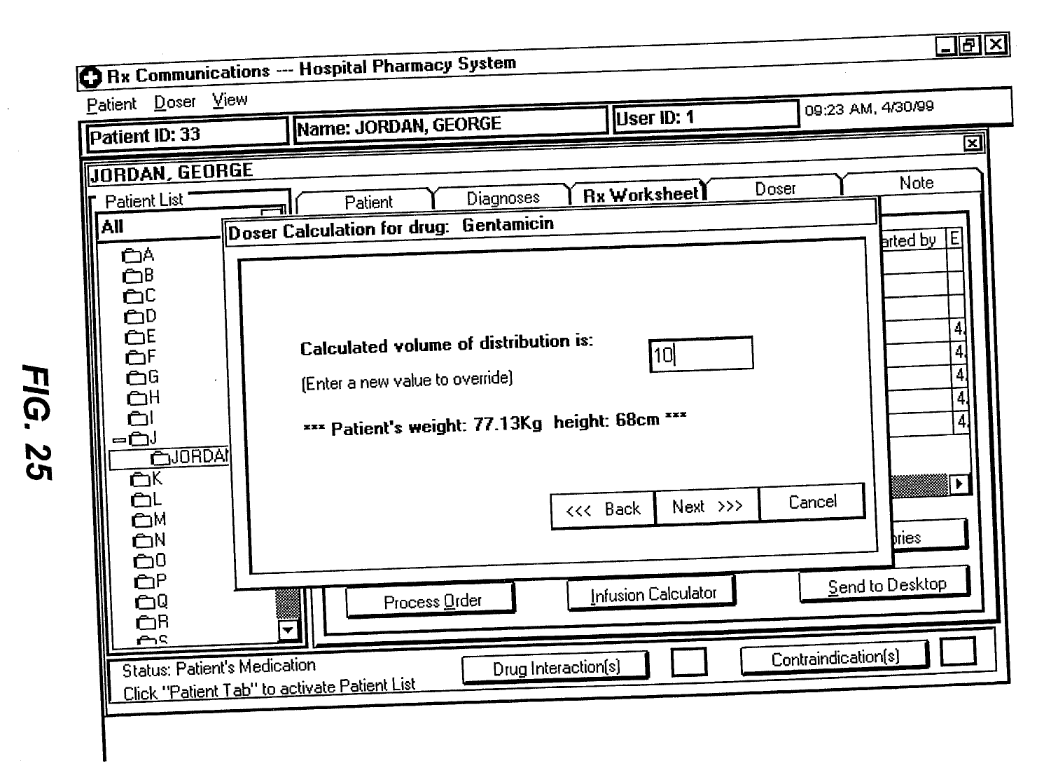

42. FIG. 25 is an illustration of an exemplary calculated volume of distribution window displayed by the KINETIC_DRUG_DOSER module of FIGS. 9A-9F;

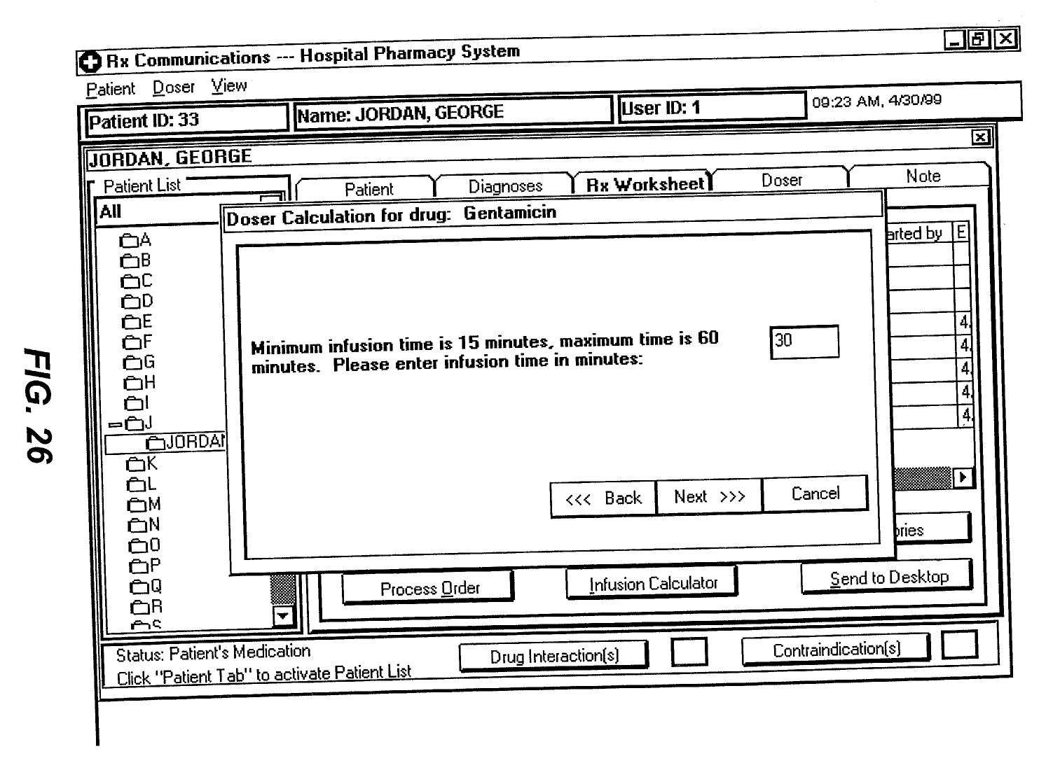

43. FIG. 26 is an illustration of an exemplary infusion time window displayed by the KINETIC_DRUG_DOSER module of FIGS. 9A-9F;

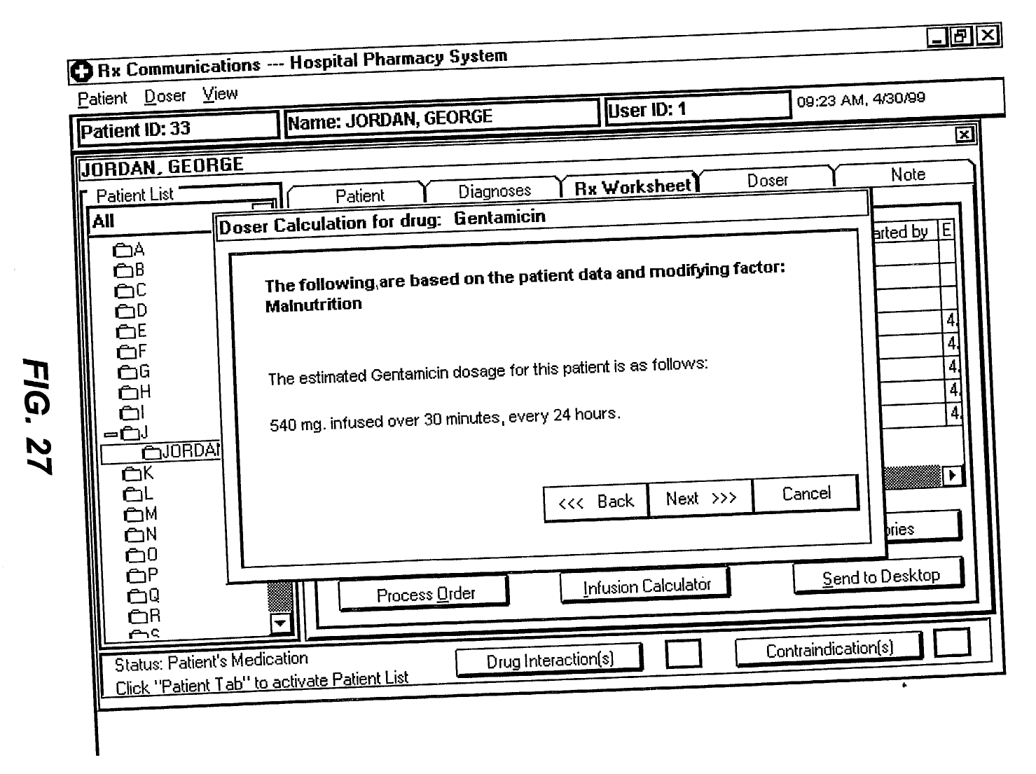

44. FIG. 27 is an illustration of an exemplary estimated dosage window displayed by the KINETIC_DRUG_DOSER module of FIGS. 9A-9F;

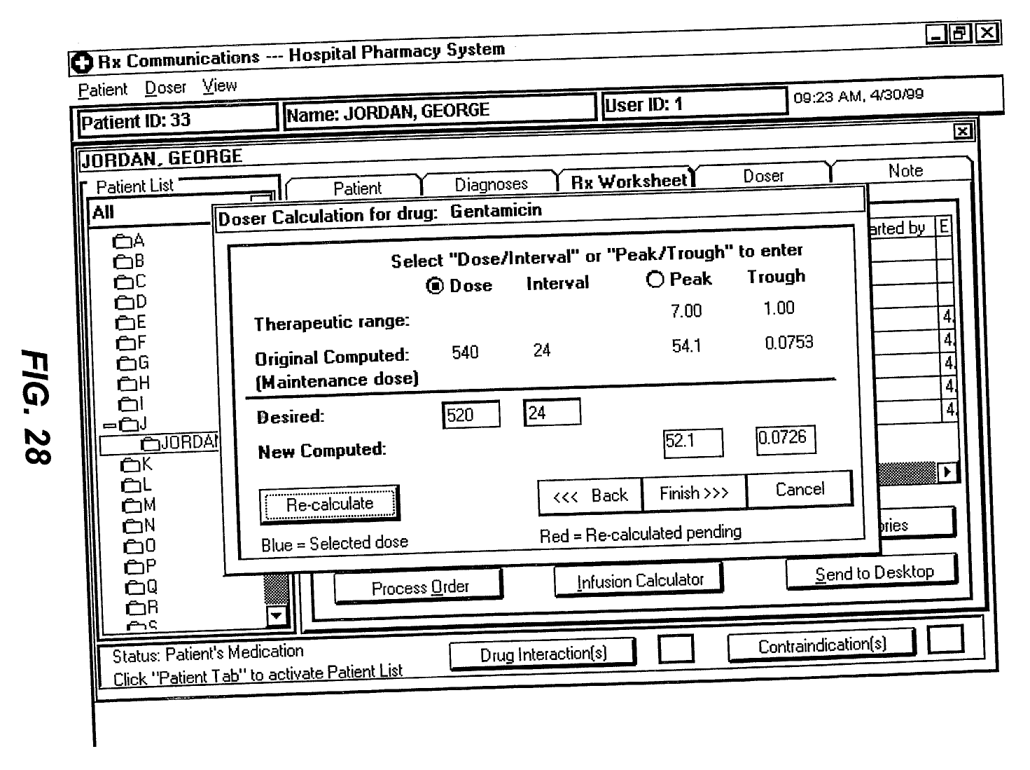

45. FIG. 28 is an illustration of an exemplary selected dose calculation window displayed by the KINETIC_DRUG_DOSER module of FIGS. 9A-9F for prospective dosing;

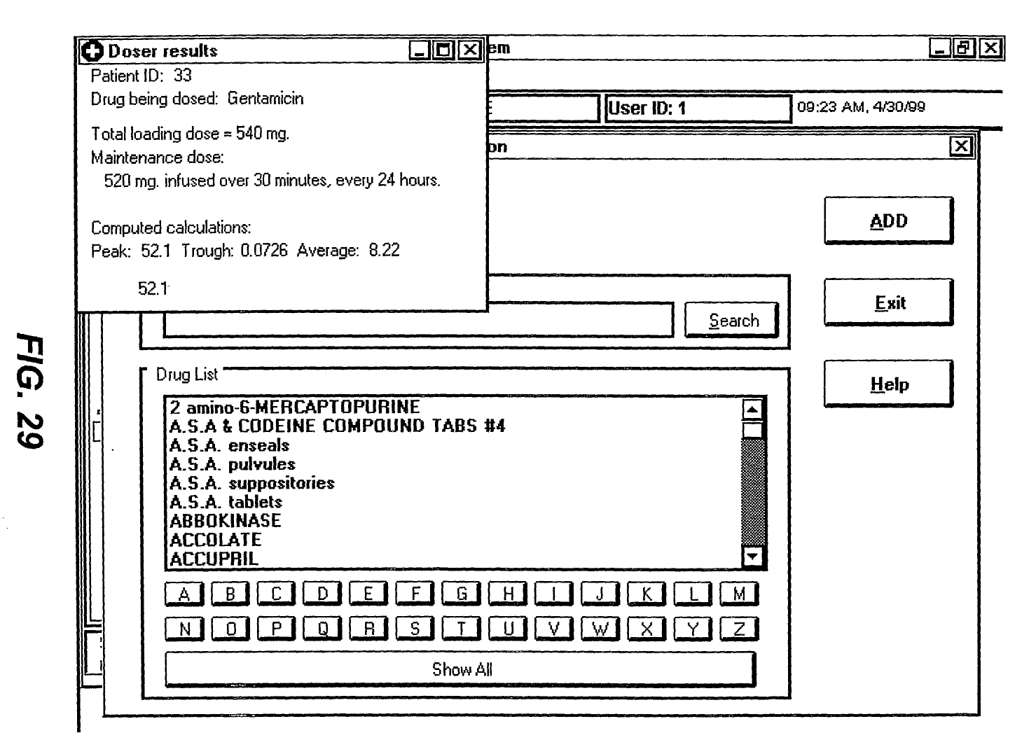

46. FIG. 29 is an illustration of an exemplary doser results window displayed by the KINETIC_DRUG_DOSER module of FIGS. 9A-9F for prospective dosing;

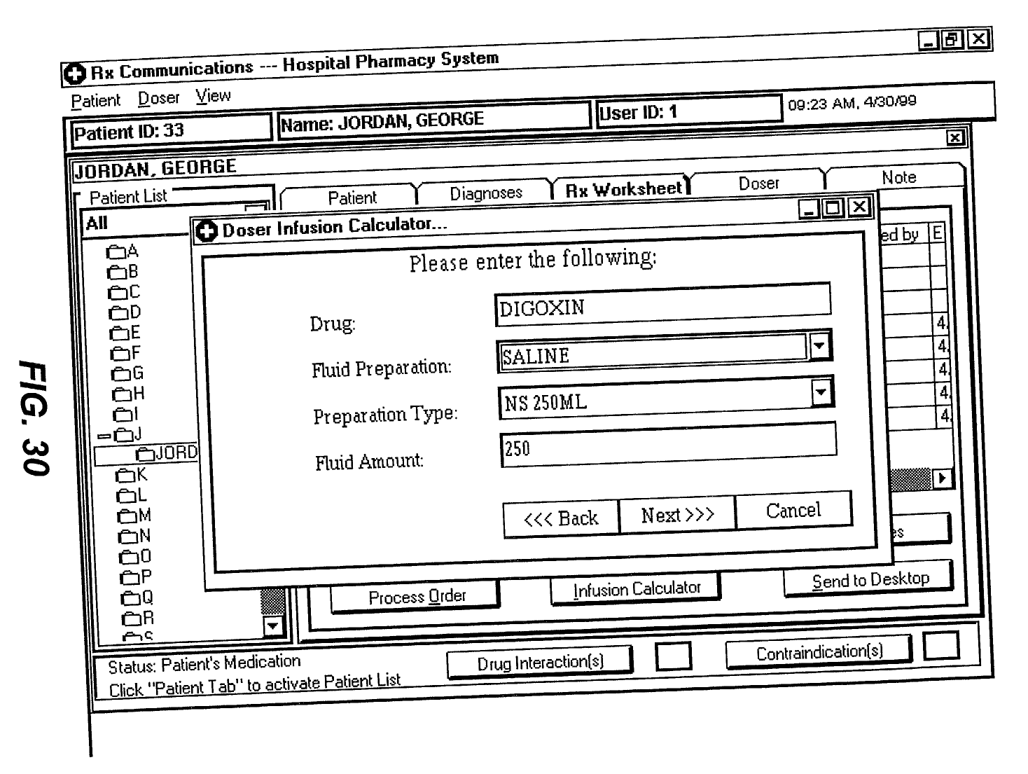

47. FIG. 30 is an illustration of an exemplary infusion calculator window displayed by the THERAPY_COORDINATOR module of FIG. 5;

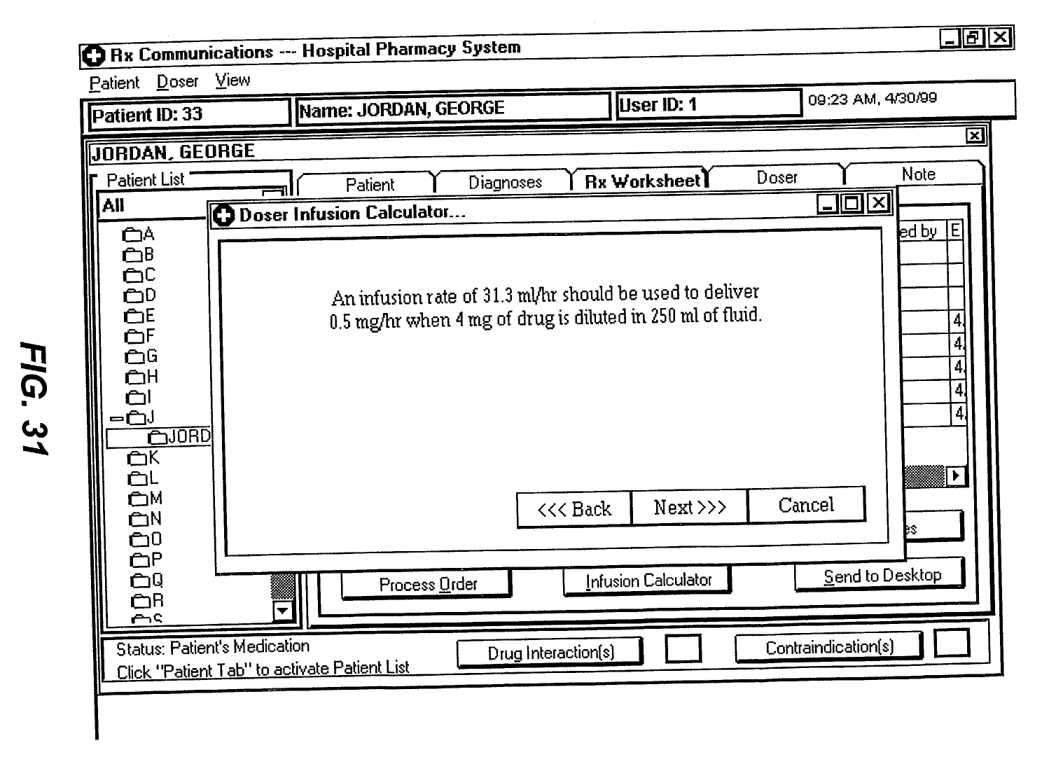

48. FIG. 31 is an illustration of an exemplary infusion calculator results window displayed by the THERAPY_COORDINATOR module of FIG. 5;

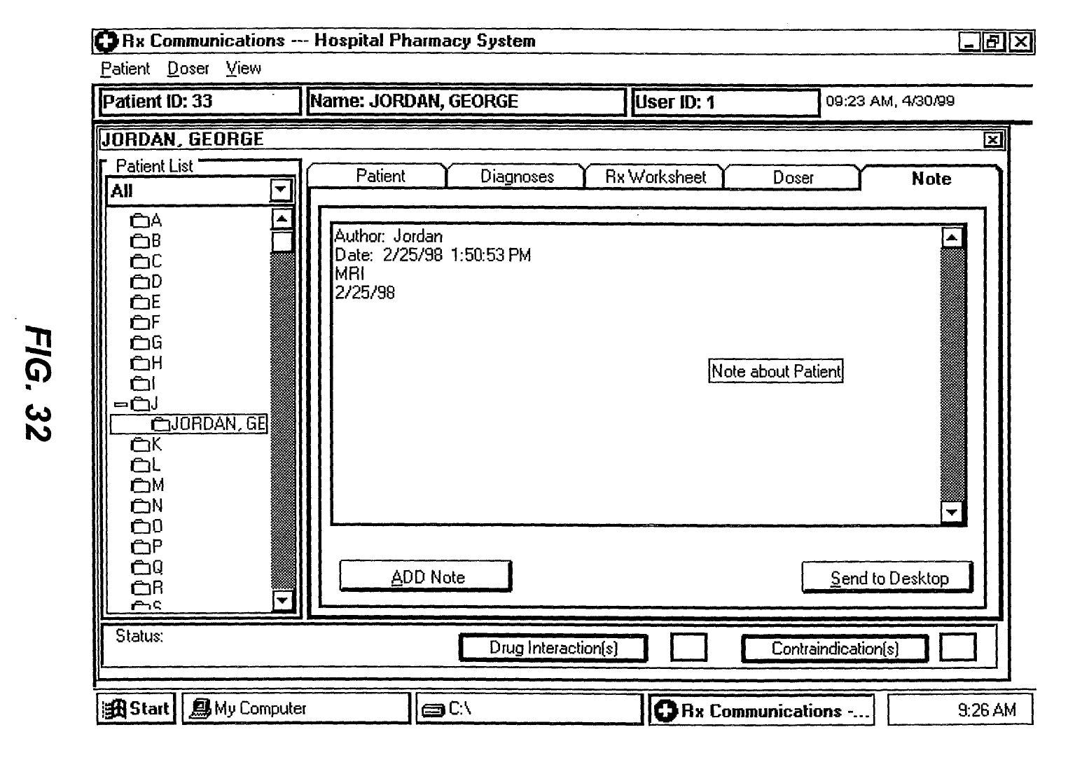

49. FIG. 32 is an illustration of an exemplary note window displayed by the THERAPY_COORDINATOR module of FIG. 5;

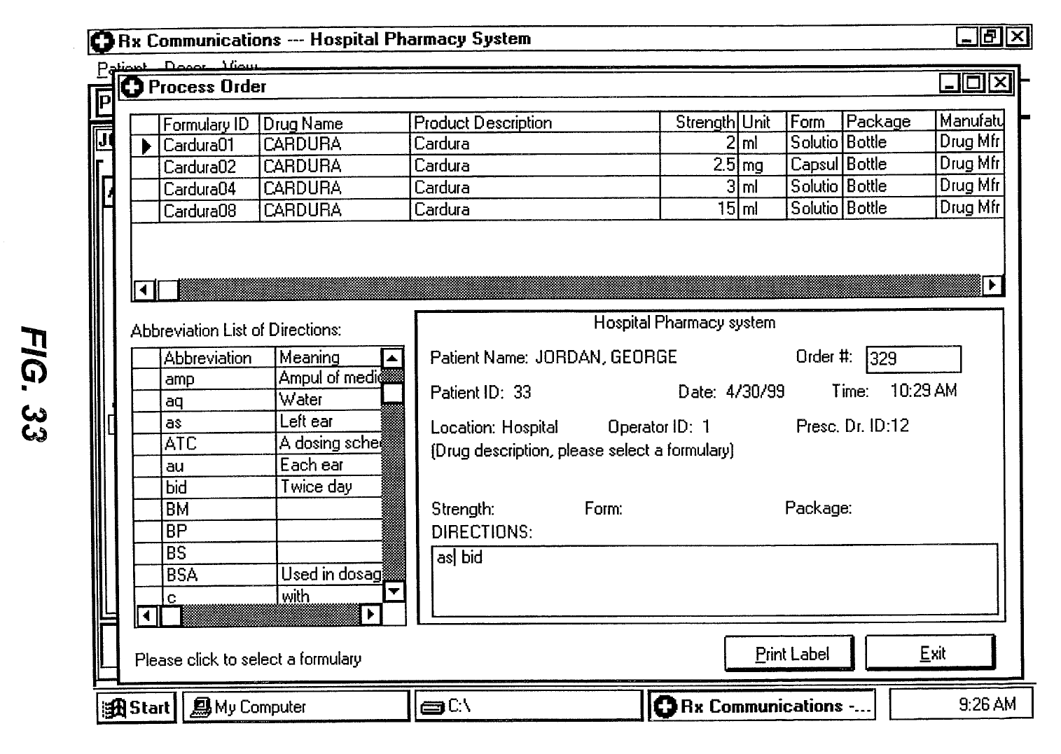

50. FIG. 33 is an illustration of an exemplary order window displayed by the ORDER_GENERATION_SYSTEM module called by the drug management process of FIG. 7;

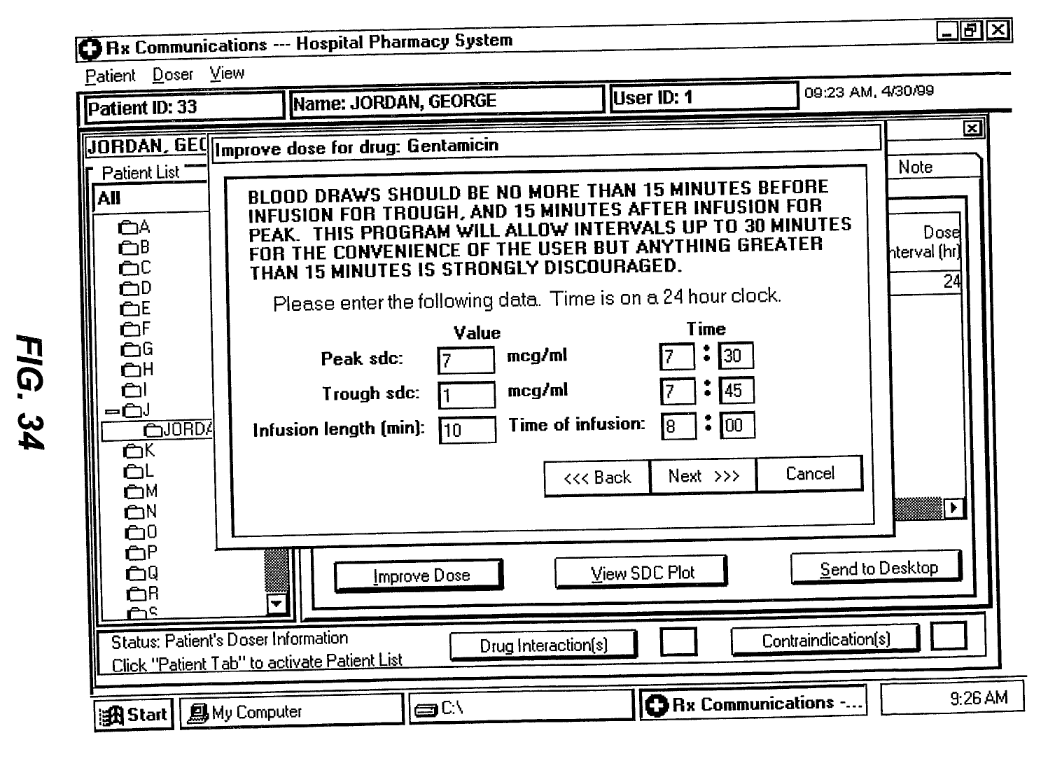

51. FIG. 34 is an illustration of an exemplary improve dose infusion entry window displayed by the KINETIC_DRUG_DOSER module of FIGS. 9A-9B;

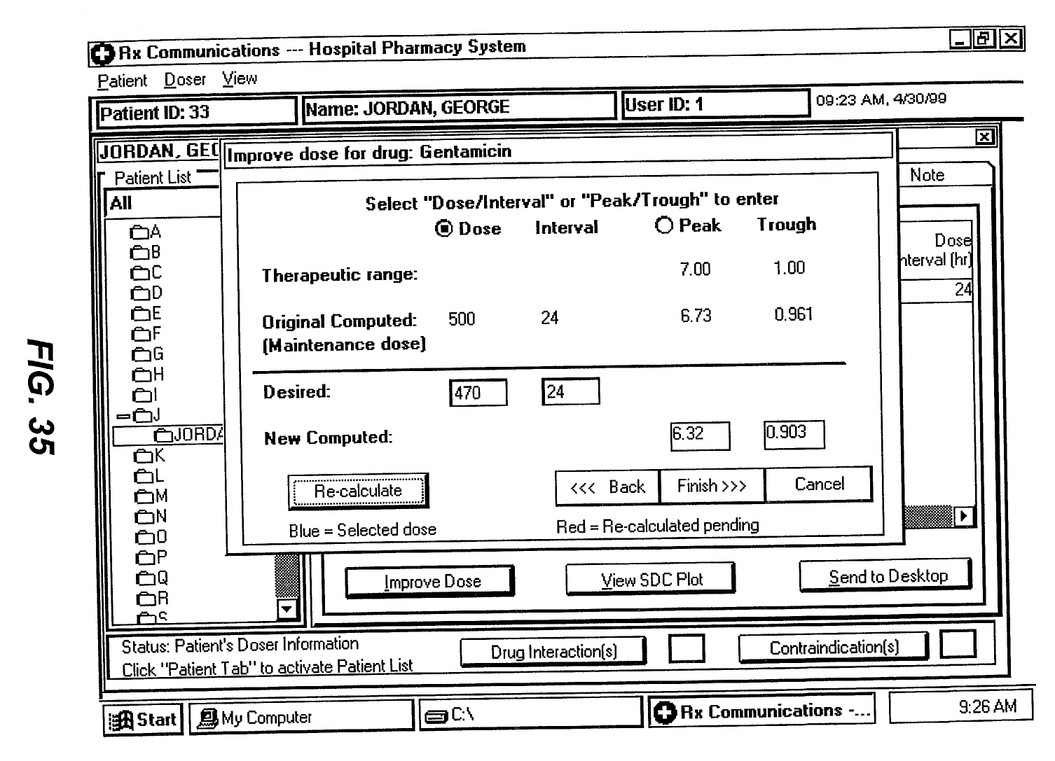

52. FIG. 35 is an illustration of an exemplary selected improve dose window displayed by the KINETIC_DRUG_DOSER module of FIGS. 9A-9F; and

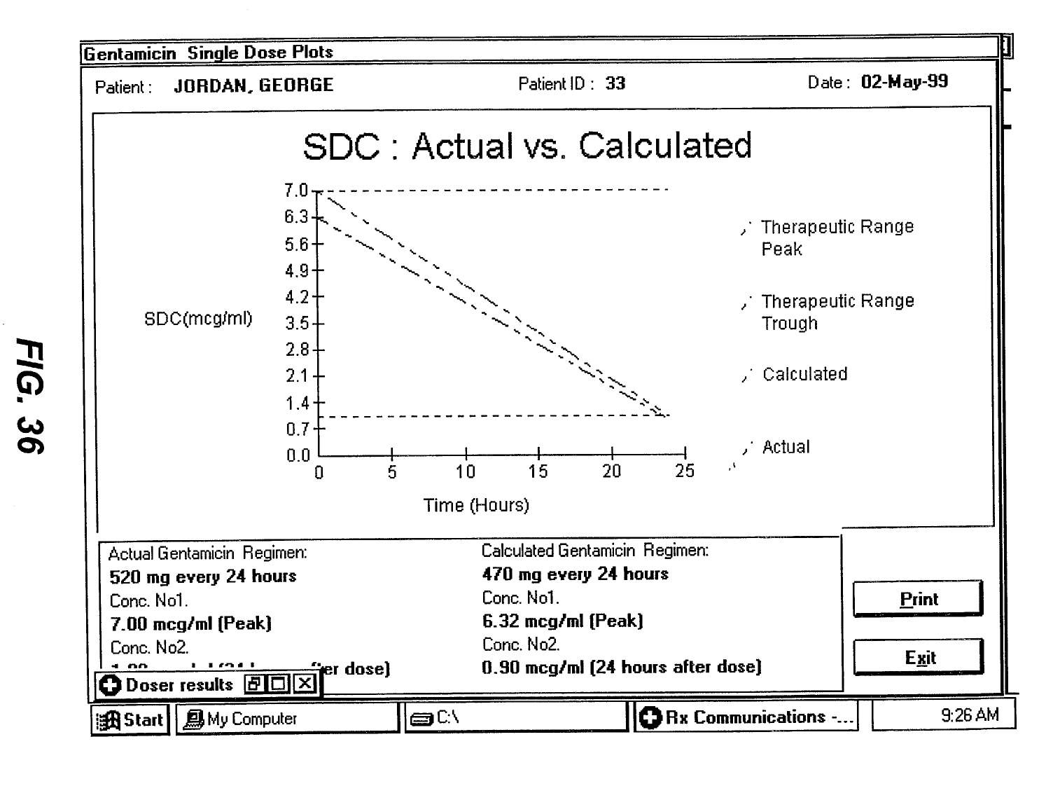

53. FIG. 36 is an illustration of an exemplary SDC plot window displayed by the KINETIC_DRUG_DOSER module of FIGS. 9A-9F.

### DETAILED DESCRIPTION OF THE PREFERRED EMBODIMENT

54. Turning now to the drawings, FIG. 1 is a block diagram of drug management software in accordance with prior art invented by Applicant. The software package 10 includes a patient data entry module 12, a drug to drug interaction module 14, a prospective dosing module 16, an improved dosing module 18, an infusion calculator 20, a patient record review module 22, and a graph display module 24. The patient data entry module 12 permits entry of patient data. Following patient data entry, a user may initiate the drug to drug interaction module 14. After a drug is entered by a user, the module 14 checks for a drug to drug interaction problem. The module 14 detects any adverse drug reaction resulting from a combination of a selected drug and a current drug of the patient. It should be understood that both the selected drug and current drug of the patient must be known by the drug management software. Next, a user may execute the prospective dosing module 16 for determining desired peak and trough blood levels for a patient. The module 16 uses pharmacokinetic equations known in the art in calculating these blood levels. If the prospective dosing stage is complete, the user has the option of executing the improved dosing module 18 for computing a dosage to achieve serum drug concentration (SDC) optimization. A user also has access at any time to the infusion calculator module 20 for computing an infusion rate for a particular drug. A user further has the option of reviewing and writing the dosing information into a patient record using the patient record review module 22 and the further option of displaying an SDC graph using the graph display module 24. This drug management software invented by Applicant and provided under the trademark THERAPY COORDINATOR™ introduced drug management software for both therapeutic drug dosing and drug to drug interaction analysis.

55. Referring to FIG. 2, a schematic block diagram of the relationship between the drug to drug interaction module 30 and associated data blocks of the drug management software 10 is shown. The drug to drug interaction module 30 uses a drug interaction file 32, a drug file 34, and a current drugs of patient file 28. The drug file 34 includes a list of available drugs, and the drug interaction file 32 includes information concerning the adverse effects of particular drug combinations. The drugs of interest in the prior art drug management software 10 were amikacin, ceftriaxone, digoxin, gentamicin, lidocaine, phenytoin, procainamide, theophylline, tobramycin, vancomycin, and warfarin. The drug to drug interaction module 30 uses the current drugs of the patient and the new drug as a search criterion for locating information within the drug interaction file 32 relevant to the patient. Any records within the drug interaction file 32 which match that search criterion are provided to the drug to drug interaction module 30.

56. Referring to FIG. 3, a flow chart of a drug dosing process performed by the prior art drug management software 10 invented by Applicant is shown. The drug dosing process 36 begins at step 38 where patient data is entered. From step 38, control proceeds to step 40 where a particular drug for the patient is then entered. Control next proceeds to step 42 where the user may enter a most recent maintenance dose. Next, in step 44, the process checks for a drug to drug interaction problem using the drug interaction file 32. In step 46, it is specifically determined if a drug interaction problem was detected. If a drug interaction problem was detected, control returns to step 40 where the user may enter a different drug. This process of detecting drug to drug interaction problems is performed by the drug to drug interaction module 30 (FIG. 2). If a drug to drug interaction problem is not detected, then control proceeds to step 48. In step 48, a loading dose and maintenance dose are computed using pharmacokinetic formulae. These calculations known in the art are performed within the prospective dosing module 16. Next, in step 50, an improved dosage is computed to optimize serum drug concentration (SDC). This operation is performed within the improved dosing module 18 (FIG. 1). From step 50, control proceeds to step 52 where a user may display a serum drug concentration graph. Display of this graph occurs within the graph display module 24 (FIG. 1). Control then proceeds to step 54 where patient data may be viewed and saved to a record. Drug dosing is completed in step 56.

57. Referring to FIG. 4, a schematic diagram of a pharmacy drug management system 90 including pharmacy drug management software 124 (FIGS. 5 and 6) in accordance with the present invention is shown. In the disclosed embodiment, the pharmacy drug management software 124 of the present invention may be stored on a pharmacy drug management compact disc (CD) 114. The computer system 100 includes a CD-ROM drive 104 for receiving the pharmacy drug management CD 114. The processor 102 of the computer system 100 serves to execute the pharmacy drug management software 124 when the pharmacy drug management CD 114 is present within the CD-ROM drive 104. Alternatively, the processor 102 may execute the pharmacy drug management software 124 from a memory 108 if the pharmacy drug management software 124 has been stored to that memory. It should be understood that other forms of media may be used external or internal to the computer system 100 for storing the pharmacy drug management software 124 of the present invention.

58. The computer system 100 further includes a modem 106 having a line for connecting to a drug company 116, a drug store 118, and a web site 120. The pharmacy drug management software 124 may communicate with the drug company 116 for reordering drugs when a drug reaches a predetermined amount. The software 124 may also electronically communicate an order for a patient directly to the drug store 188. The web site 120 includes a patient data matching database 122. The patient data matching database 122 may be accessed at any time by a patient data matching module 130 (FIG. 5) of the pharmacy drug management software 124. The computer system 100 further includes a display screen 110 for communicating information to a pharmacist or doctor and a printer 112 for receiving reports generated by a report writer module 134 (FIG. 5) of the software 124. One type of report which may be generated and printed is a report, known as a DUE, indicating the utilization of a drug. The report may also indicate the top drugs prescribed and which doctors are writing orders resulting in adverse drug reactions. The computer system 100 may be of any type, such as desktop, laptop, or handheld, and may be for standalone or network use. It is contemplated that the pharmacy drug management system of the present invention may be used at any location in the world to control iatrogenic illnesses, such as a hospital, nursing home, HMO, or a home healthcare or home infusion business.

59. Referring to FIG. 5, a schematic block diagram of the modules of the pharmacy drug management software 124 is shown. The pharmacy drug management software 124 includes a drug dosing module 126, a therapy management module 128 termed the THERAPY_COORDINATOR, an order generation module 132, and the report writer module 134. The THERAPY_COORDINATOR module 128 provides a drug interaction analysis sub-module 150. The drug interaction analysis sub-module 150 is used to detect and correct drug interaction problems. These drug interaction problems not only include drug to drug interaction problems, but also problems based on interactions between a drug and a medical condition, allergy, or diet of a patient. The results obtained by the drug interaction analysis sub-module 128 may be provided to the report writer 134. The drug interaction analysis sub-module 150 fetches medical data for the patient from a list of the patients' medical conditions.

60. In accordance with the present invention, the drug dosing module 126 is used for drugs having a narrow therapeutic index. The module 126 provides both prospective and improved dosing of drugs within this category. In the disclosed embodiment, the drugs for which information is provided include aminoglycasides, strong antibiotics, cephlosporins, cardiovascular disease drugs, and pulmonary disease drugs, to name a few. These particular drugs require both careful dosing and continuous monitoring. A drug dosed by the drug dosing module 126 may be entered into an order using the order generation module 132. The order generation module 132, which is termed the ORDER_GENERATION_SYSTEM, provides an on-screen order to a doctor or pharmacist. By using the pharmacy drug management software 124 to write an order after the software 124 has checked for drug interaction problems, the need to track down a doctor to address a drug interaction problem after generating an order is eliminated.

61. Referring to FIG. 6, a schematic block diagram representing use of the drug interaction analysis sub-module 150 is shown. With respect to the patient, the drug interaction analysis sub-module 150 receives patient physical data 138, clinical lab reports 140, the current drugs of the patient 142, the medical condition of the patient 144, the patient internal chart 146, and the diet of the patient 148. The drug interaction analysis sub-module 150 utilizes each of these forms of patient medical data in determining whether a drug interaction problem exists. To detect whether a drug interaction problem exists, the drug interaction analysis sub-module 150 uses the patient medical data, drugs, and the new drug as a search criterion for searching a drug information file 154 and a medical condition file 152. While the medical condition file 152 contains information concerning interactions between drugs and medical conditions, the drug information file 154 contains information concerning a drug-drug interaction or a drug-food interaction. It should be understood that a food or drug allergy of a patient may contribute to a drug-drug interaction or drug-food interaction. The drug file 156 contains a list of the available drugs.

62. Referring to FIG. 7A, the drug management process performed by the THERAPY_COORDINATOR module 128 begins at step 160 where a doctor or pharmacist ID is entered. Next, in step 162, a patient ID is then entered. FIG. 13 depicts an exemplary window for such entry. From step 162, control proceeds to step 164 where patient data is then entered. In the disclosed embodiment, patient data includes a patient ID, a doctor ID, a location of the patient, a room number of the patient, a name of the patient, a date of birth, a height of the patient, and a weight of the patient as illustrated by the exemplary patient window in FIG. 14. From step 164, the doctor or pharmacist may proceed to step 166, 168, 170 or 174. At step 166, the doctor or pharmacist is able to add a drug for the selected patient. FIG. 15 depicts an exemplary “add medication” window for such an entry. In step 168, the doctor or pharmacist is able to add a medical condition for the selected patient. In the disclosed embodiment, a medical condition may be selected by searching for a medical condition by the ICD-9 class number of the medical condition, the ICD-9 subclass number of the medical condition, or the name of the medical condition as illustrated by the “add medical condition” window of FIG. 20. ICD-9 codes and subcodes are used in the United States to identify medical diagnoses. From either step 166 or step 168, control proceeds to step 176 where the drug interaction analysis sub-module 150 is called.

63. Referring to FIGS. 8A-8B, flow charts of the process performed by the drug interaction analysis sub-module 150 is shown. The sub-module 150 begins at step 184 where the new drug selected for the patient is compared with the current drugs of the patient. Control next proceeds to step 186 where the new drug is then compared with the food included in the diet of the patient. From step 186, control proceeds to step 188 where the new drug is compared with the medical conditions of the patient. Control then proceeds to step 190 where the new drug is compared with the allergies (food and drug) of the patient. Each of the above compare operations is performed in the manner discussed in connection with FIG. 6. Next, in step 192, it is determined whether there is a drug interaction problem for the particular patient. If there is no drug interaction problem, control proceeds to step 194 where an indication that a drug interaction problem is not present is provided. From step 194, control proceeds to step 204. If a drug interaction problem is detected, control proceeds to step 196 where a warning is provided. If the detected problem is an interaction between drugs, a “drug interaction” warning is provided. If the detected problem is an interaction between the new drug and a medical condition of the patient, a contraindication warning is provided. FIG. 16 depicts an exemplary “drug interaction” warning window for an interaction between lanoxin and eythromycin, and FIG. 17 depicts an exemplary contraindication warning window for an interaction between vancomycin and pneumonia. In the disclosed embodiment, a small box on the display screen 110 for “drug interactions” goes from green to red if a drug causing a “drug interaction” warning is selected. If a drug causing a contraindication warning is selected, a small box for contraindications goes from green to red. Either box returns to green if the respective drug is deselected. Also, in the disclosed embodiment, a warning window includes a representation of a traffic light which blinks red. From step 196, control proceeds to step 198 or to step 200. In step 198, the sub-module 150 recommends alternative drugs to the currently selected drug. From step 198, control proceeds to step 200. In step 200, the doctor or pharmacist acknowledges the warning provided. Next, in step 202, the sub-module 150 requests an indication as to whether the warning should be overrided. If the doctor or pharmacist overrides the warning, control proceeds to step 204 where the drug is selected. If the doctor or pharmacist does not override the warning, then control proceeds to step 206 where an alternative drug may be entered. From step 204 and 206, control proceeds to step 208 where drug interaction analysis by the sub-module 150 is completed.

64. From step 176, control proceeds to step 178 where the drug management process determines if the selected drug is a doser drug. FIG. 22 depicts an exemplary doser drug detection window for requesting an indication from the doctor or pharmacist as to whether a doser calculation is desired. In accordance with the present invention, a doser drug is any drug having a narrow therapeutic index. If the selected drug is not a doser drug, control returns through connector B (FIGS. 7A-7B). If the drug selected is a doser drug and the user elects to dose the particular drug with the doser, control proceeds to step 180 where the KINETIC_DRUG_DOSER module 126 (“doser”) is called.

65. Referring to FIG. 9A, the KINETIC_DRUG_DOSER module 126 begins at step 212 where the height and weight of the patient is confirmed. Next, in step 214 it is determined whether a creatinine clearance for the patient is known. A creatinine clearance represents the fluid in a patient's blood created by muscle mass. The creatinine clearance is used to calculate the rate of absorption, rate of distribution, rate of metabolism, and rate of excretion of a drug in relation to the patient. If the creatinine clearance for the patient is known, then control proceeds to step 220 where the creatinine clearance is entered. If the creatinine clearance for the patient is not known, then control proceeds to step 216 where particular values are entered for calculating the creatinine clearance for the patient. In the disclosed embodiment, either two serum creatinine measurements and the number of days between the measurements is entered or urine volume, creatinine concentration, and serum creatinine draws at midpoint of collection is entered. Next, in step 218, the creatinine clearance is calculated. From step 220 and step 218, control proceeds to step 222 where the user indicates whether the patient is neonatal, pediatric, or adult. From step 222, control proceeds to step 224 where a volume of distribution and infusion time of the patient is entered. The volume of distribution is a number representing the area in the patient where the drug will be distributed to treat the medical condition. Next, in step 226, the modifying factors of the patient are entered as depicted by the exemplary modifying factors window of FIG. 23 for the drug gentamicin. Modifying factors represent conditions of the patient which impact the doser calculation. For example, in the disclosed embodiment, the modifying factors for the drug gentamicin include dehydration, overhydration, severe burns, ascities, and malnutrition. The module 126 also may provide a dosing guideline specific to the selected modifying factor as illustrated by the exemplary malnutrition window of FIG. 24. Next, in step 230, a patient kinetic constant, half-life, and loading dose are generated. A patient kinetic constant is a fixed number used for pharmacokinetic calculations that is specific to a patient. A half-life is the amount of time in hours for which a drug will last in a patient. FIG. 25 shows an exemplary calculated volume of distribution window. These values generated in step 230 are calculated using pharmacokinetic equations known in the art. These equations are provided in the book, Winter, M. E., “Basic Clinical Pharmacokinetics,” 3rd Edition, Applied Therapeutics, Inc., Vancouver, W. A. 1994, which is incorporated herein by reference as if set forth in its entirety. In accordance with the present invention, the pharmacokinetic equations used are specific to the particular classes of the medical conditions of the patient and to the particular classes of drugs of the patient.

66. From step 230, control proceeds to step 232 where an estimated dosage is calculated. An exemplary estimated dosage window for the drug gentamicin is shown in FIG. 27. From step 232, control proceeds to step 236 or step 234. The doctor or pharmacist has the option of entering either a peak and trough in step 236 or the option of entering a maintenance dose and an interval in step 234 as illustrated by the exemplary selected doser calculation window of FIG. 28. If a peak and trough are entered in step 236, control then proceeds to step 238 where a maintenance dose and interval are generated by module 126. If a maintenance dose and interval are entered in step 234, then control proceeds to step 240 where a peak and trough are calculated by the module 126. Either input process may be repeated continuously until a satisfactory set of values are achieved. Next, in step 242, it is determined whether the generated and entered data is to be saved. If the doctor or pharmacist indicates that the data is not to be saved, then control returns to either step 236 or step 234. If the doctor or pharmacist elects to save the data, then control proceeds to step 244 where the doser results are displayed as illustrated by the exemplary doser results window of FIG. 29. That window provides the patient ID, the drug being dosed, the total loading dose, the maintenance dose, the maintenance dose, the computed peak, computed trough, and computed average. From step 244, the drug dosing process terminates in step 246.

67. The doctor or pharmacist also has the alternative to proceed from steps 238 and 240 to step 324 representing entry into an improved dosing stage of the dosing process (FIG. 9D). From step 324 control proceeds to step 326 where the patient height and weight are confirmed. Control next proceeds to step 328 where it is determined if the creatinine clearance for the patient is known. If the creatinine clearance is not known, then control proceeds to step 330 where values are inputted for calculating the creatinine clearance. Control next proceeds to step 332 where the creatinine clearance is calculated. If the creatinine clearance for the patient is known, then control proceeds to step 334 where the creatinine clearance is entered. From step 332 and step 334, control proceeds to step 336 where it is determined if the patient is neonatal, pediatric or adult. From step 336 control proceeds to step 338 where it is determined if the doctor or pharmacist desires to change the maintenance dose for the patient. If the doctor or pharmacist indicates that a change is desirable, then control proceeds to step 340 where a new maintenance dose is entered. From step 340 and from step 338, if a change of the maintenance dose is not selected, control proceeds to step 342. In this step, a doctor or pharmacist is able to enter values and times for a peak blood draw and trough blood draw, an infusion length, and a time of infusion as illustrated by the exemplary improved dose calculation window of FIG. 34.

68. Control then proceeds to step 344 where the volume of distribution is generated from the values provided in step 342. From step 344, control may proceed to either step 346 or step 350. In step 346, a desired peak and trough may be entered, and in step 350, a desired dose and interval may be entered. From step 346, control proceeds to step 348 where a new dose and interval are generated. From step 350, control proceeds to step 356 where a new dose and interval are generated. Steps 346, 348, 350, and 356 are represented by the exemplary improved dose calculation window depicted in FIG. 35. From step 348 and step 356, control proceeds to step 358 where it is determined if the doctor or pharmacist desires to save the data. The doctor or pharmacist also has the options from steps 348 and 356 to repeat the process of entering the desired parameters in steps 346 or 350. If the doctor or pharmacist indicates that the data is not to be saved, then control proceeds again to either step 346 or step 350. If an indication is provided that data is to be saved, then control proceeds to step 360 where a serum drug concentration (SDC) plot may be viewed or to step 362 where the improved dosing process is completed. A serum drug concentration represents a level of drug that will remain constant through doses of a drug for a patient. The SDC plot represents the calculated SDC, the actual SDC, and the therapeutic range peak and trough as illustrated by the exemplary SDC plot window of FIG. 36. From step 360, control terminates through step 362.

69. Returning to FIG. 7A, an ARCHIVE_DATABASE_SYSTEM module 248 may be called in step 170. In the disclosed embodiment, the ARCHIVE_DATABASE_SYSTEM module 248 accesses a relational database 122 for matching a current patient's medical condition to patient therapies stored within the database 122. Referring to FIG. 10, beginning in step 250, the specific patient data of interest is entered as search criteria. Control then proceeds to step 252 where the database 122 is searched for matches with this search criteria. In this way, a clinical professional can locate previous patient therapies that have been administered for treatment of medical conditions similar to a current patient. Control next proceeds to step 254 where matches from the database 122 are returned along with the associated data. Referring to FIG. 11, an exemplary record 257 is shown including an upper portion which is preferably searchable and a lower portion 274 associated with the upper portion 258. The upper portion 258 includes a field 262 for storing an age of a patient, a field 264 for storing the race of a patient, a field 266 for storing a sex of a patient, a field 268 for storing the infection of a patient, a field 270 for storing the site of the infection of the patient, and a field 272 for storing the treatment of the patient. The lower portion 274 includes a field 276 for storing bacteriological reports of a patient, a field 278 for storing clinical reports of a patient, and a field 280 for storing urine sample reports for a patient. The process of matching patient data with the records contained in the database is completed through step 256.

70. Referring to FIG. 12, a block diagram of the features accessible from and through the THERAPY_COORDINATOR module 128 is shown. A note feature 300 permits a doctor or pharmacist to maintain a record of events, such as consultations and non-medical intervention, for a particular patient as illustrated by the exemplary note window of FIG. 32. In the disclosed embodiment, patient events may be typed directly into a note window or may be entered into the note window using a pen and a writing tablet (not shown) connected to the computer system 100. A help file feature 302 provides information to a doctor or pharmacist concerning a particular drug or medical condition. In this way, a doctor or pharmacist need not leave the display section 110 to access such information. A patient feature 304 allows for entry and editing of patient data. A formulary feature 306 provides a list of available drugs readily accessible to the doctor or pharmacist. An infusion calculator 308 for calculating infusion rates is also provided by the module 128 as illustrated by the exemplary infusion calculator windows of FIGS. 30-31. A patient matching feature 310 renders the patient matching database 122 described above readily available upon command. An Rx worksheet feature 312 provides a list of the drugs for the particular patient along with a start date, end date, date the order is written, date inventory is verified, and the date the order is filled for each drug as illustrated by the exemplary Rx Worksheet window of FIG. 19. An advisories feature 314 provides a doctor or pharmacist with some advisories that are specific to a particular drug and other advisories that are specific to the class of patient (neonatal, pediatric, and geriatric). FIG. 21 depicts an exemplary advisory window for the drug vancomycn. A doser function 316 provides for the KINETIC_DRUG_DOSER module as described above. A contradictions feature 318 and a drug interactions feature 322 permit a doctor or pharmacist at any time to check the current list of contradictions and “drug interactions.” For example, a list of contradictions is depicted by the exemplary contradictions list window of FIG. 18. A diagnosis feature 320 provides for reviewing and adding medical conditions for a particular patient.

71. In accordance with the present invention, a doctor or pharmacist knows about drug interaction problems before writing an order, eliminating the problem of locating a doctor when a drug interaction problem is discovered for an order already written by the doctor. The pharmacy drug management software of the present invention also permits doctors and pharmacists to view advisories, drug and medical condition information help files, patient data, and orders so that a doctor or pharmacist need not leave the screen of the computer system 100 to properly diagnose patients. Further, as a drug is added for a patient, the present invention automatically detects whether a drug is a doser drug needing precise therapeutic dosing and automatically checks for drug interaction problems based on the patient data, reducing the likelihood of clinical misjudgments and clinical liability for adverse drug reactions. The present invention thus overall provides a total drug care system.

72. It should be understood that the exemplary windows illustrated herein are not exhaustive of the windows which are provided by the present invention. Further, it should be understood that the specific pharmacokinetic equations, medical conditions, and drugs in the disclosed embodiment may be varied in accordance with the present invention.

73. The foregoing disclosure and description of the invention are illustrative and explanatory thereof, and various changes in the number of variables, number of parameters, order of steps, field sizes, data types, code elements, code size, connections, components, and materials, as well as in the details of the illustrated hardware and software and construction and method of operation may be made without departing from the spirit of the invention.

What is claimed is:

1. A computer system adapted for patient specific drug dosing, drug interaction analysis and order generation, comprising:
a processor;
a medium readable by the processor for storing patient specific drug dosing code, drug interaction analysis code, and order entry code;
the patient specific drug dosing code when executed causing the processor to perform the steps of:
receiving medical data for a patient;
receiving an indication of a new drug for the patient;
detecting if the new drug provides a narrow therapeutic index;
receiving a first set of dosing parameters for the patient if the new drug provides a narrow therapeutic index; and
calculating a second set of dosing parameters for the patient from the medical data of the patient and the first set of dosing parameters;
the drug interaction analysis code when executed causing the processor to perform the steps of:
receiving medical data for the patient;
receiving an indication of a new drug for the patient;
checking the new drug for a drug interaction problem;
indicating a drug interaction problem if a drug interaction problem is detected;
selecting an alternative drug for the patient if the alternative drug is provided; and
selecting the new drug for the patient if an override command is received; and
the order generation code when executed causing the processor to perform the steps of:
providing an order on a display screen of the computer system after the drug interaction analysis code is executed;
receiving order information for the new drug into the order; and
processing the order.

2. The computer system of claim 1, wherein the first set of dosing parameters for the patient comprise a creatinine clearance, a volume of distribution, modifying factors, infusion time, and dosing interval.

3. The computer system of claim 1, wherein the second set of dosing parameters comprise a patient kinetic constant, a half-life, and a dosage of the drug.

4. The computer system of claim 1, the patient specific drug dosing code when executed causing the processor to perform the further steps of:
receiving a desired dose of the drug; and
calculating a peak drug value for the patient and a trough drug value for the patient from the desired dose of the drug, the first set of dosing parameters, the second set of dosing parameters, and the medical data for the patient.

5. The computer system of claim 1, the patient specific drug dosing code when executed causing the processor to perform the further steps of:
entering an improved dosing stage;
receiving a third set of dosing parameters for the patient;
calculating a fourth set of dosing parameters for the patient from the medical data for the patient and the third set of dosing parameters.

6. The computer system of claim 5, wherein the third set of dosing parameters comprise a maintenance dose, a creatinine dosing, a peak blood draw, a trough blood draw, an infusion length, a time of the peak blood draw, a time of the trough blood draw, and an infusion time.

7. The computer system of claim 5, wherein the fourth set of dosing parameters comprise a volume of distribution.

8. The computer system of claim 1, the patient specific drug dosing code when executed causing the processor to perform the further steps of:
receiving a peak drug value for the patient and a trough drug value for the patient; and
calculating a maintenance dose of the drug and a maintenance interval of the drug from the peak draw of value for the patient and the trough drug value for the patient.

9. The computer system of claim 1, the patient specific drug dosing code when executed causing the processor to perform the further steps of:
receiving a maintenance dose of the drug and a maintenance interval of the drug; and
calculating the peak drug value for the patient and the trough drug value for the patient from the maintenance dose of the drug and the maintenance interval of the drug.

10. The computer system of claim 1, the receiving medical data step for the drug interaction analysis code comprising the step of:
receiving data regarding drugs of the patient; and
the checking step comprising the step of:
checking the new drug for a drug interaction problem based on an interaction between the new drug and the drugs of the patient.

11. The computer system of claim 1, the receiving medical data step for the drug interaction analysis code comprising the step of:
receiving data regarding medical conditions of the patient; and
the checking step comprising the step of:
checking the new drug for a drug interaction problem based on an interaction between the new drug and medical conditions of the patient.

12. The computer system of claim 1, the receiving medical data step for the drug interaction analysis code comprising the step of:
receiving data regarding a diet of the patient; and
the checking step comprising the step of:
checking the new drug for a drug interaction problem based on an interaction between the new drug and the diet of the patient.

13. The computer system of claim 1, the receiving medical data step for the drug interaction analysis code comprising the step of:
receiving data regarding allergies of the patient; and
the checking step further comprising the step of:
checking the new drug for a drug interaction problem based on an interaction between the drug and the allergies of the patient.

14. The computer system of claim 1, the receiving medical data step for the drug interaction analysis code comprising the step of:
receiving data regarding drugs of the patient; and
the checking step comprising the step of:
searching a drug file containing information regarding drug to drug interaction problems with the new drug and the drugs of the patient as a search criterion.

15. The computer system of claim 1, the receiving medical data step for the drug interaction analysis code comprising the step of:
receiving data regarding drugs of the patient; and
the checking step comprising the step of:
searching a medical condition file containing information regarding drug interaction problems with the new drug and the medical conditions of the patient as a search criterion.

16. The computer system of claim 1, the receiving medical data step for the drug interaction analysis code comprising the step of:
receiving data regarding the drugs of the patient; and
the checking step comprising the step of:
searching a file containing information regarding the drug interaction problems based on allergies with the new drug and the allergies of the patient as a search criterion.

17. The computer system of claim 1, the receiving medical data step for the drug interaction analysis code comprising the step of:
receiving data regarding the drugs of the patient; and
the checking step comprising the step of:
searching a file containing information regarding drug interaction problems based on food with the new drug and the food of the patient as a search criterion.

18. The computer system of claim 1, the computer system further comprising:
a printer; and
the order generation code when executed causing the processor to perform the further step of:
sending the order to the printer as a label for the drug or a prescription for the patient.

19. The computer system of claim 1, the medium readable by the processor being a compact disc, the computer system further comprising:
a CD-ROM drive for receiving the compact disc storing the patient specific drug dosing code, the drug interaction analysis code, and the order generation code.

20. The computer system of claim 1, wherein the medium readable by the processor for storing patient specific drug dosing code, drug interaction, analysis code, and order entry code is a memory.

21. The computer system of claim 1, the medium readable by the processor further storing:
patient data matching code for searching a patient data matching database for therapy profiles of patients with medical data matching medical data for the patient.

22. The computer system of claim 21, wherein the patient data matching database is located on a website.

23. A method of patient specific dosing of a drug with a narrow therapeutic index using a comprehensive drug management program executed by a computer system, comprising the steps of:
receiving medical data for a patient;
receiving an indication of a new drug for the patient;
detecting if the new drug provides a narrow therapeutic index;
receiving a first set of dosing parameters for the patient if the new drug provides a narrow therapeutic index; and
calculating a second set of dosing parameters for the patient from the medical data of the patient and the first set of dosing parameters.

24. The method of claim 23, wherein the first set of dosing parameters for the patient comprise a creatinine clearance, a volume of distribution, and modifying factors, infusion time, and dosing interval.

25. The method of claim 23, wherein the second set of dosing parameters comprise a patient kinetic constant, a half-life, and a dosage of the drug.

26. The method of claim 23, further comprising the steps of:
receiving a desired dose of the drug; and
calculating a peak drug value for the patient and a trough drug value for the patient from the desired dose of the drug, the first set of dosing parameters, the second set of dosing parameters, and the medical data for the patient.

27. The method of claim 23, further comprising the steps of:
entering an improved dosing stage;
receiving a third set of dosing parameters for the patient;
calculating a fourth set of dosing parameters for the patient from the medical data for the patient and the third set of dosing parameters.

28. The method of claim 27, where in the third set of dosing parameters comprise a maintenance dose, a creatinine dosing, a peak blood draw, a trough blood draw, an infusion length, a time of the peak blood draw, a time of the trough blood draw, and an infusion time.

29. The method of claim 27, wherein the fourth set of dosing parameters comprise a volume of distribution.

31. The method of claim 23, the patient specific drug dosing code when executed causing the processor to perform the further steps of:
receiving a peak drug value for the patient and a trough drug value for the patient; and
calculating a maintenance dose of the drug and a maintenance interval of the drug from the peak draw of value for the patient and the trough drug value for the patient.

32. A method of detecting and correcting a drug interaction problem using a comprehensive drug management program executed by a computer system before ordering a drug for a patient, comprising the steps of:
receiving medical data for the patient;
receiving an indication of a new drug for the patient;
checking the new drug for a drug interaction problem;
indicating a drug interaction problem if a drug interaction problem is detected;
selecting an alternative drug for the patient if the alternative drug is provided; and
selecting the new drug for the patient if an override command is received.

33. The method of claim 32, the receiving medical data step for the drug interaction analysis code comprising the step of:
receiving data regarding drugs of the patient; and
the checking step comprising the step of:
checking the new drug for a drug interaction problem based on an interaction between the new drug and the drugs of the patient.

34. The method of claim 32, the receiving medical data step for the drug interaction analysis code comprising the step of:
receiving data regarding medical conditions of the patient; and
the checking step comprising the step of:
checking the new drug for a drug interaction problem based on an interaction between the new drug and medical conditions of the patient.

35. The method of claim 32, the receiving medical data step for the drug interaction analysis code comprising the step of:
receiving data regarding a diet of the patient; and
the checking step comprising the step of:
checking the new drug for a drug interaction problem based on an interaction between the new drug and the diet of the patient.

36. The method of claim 32, the receiving medical data step for the drug interaction analysis code comprising the step of:
receiving data regarding allergies of the patient; and
the checking step further comprising the step of:
checking the new drug for a drug interaction problem based on an interaction between the drug and the allergies of the patient.

37. The method of claim 32, the receiving medical data step for the drug interaction analysis code comprising the step of:
receiving data regarding drugs of the patient; and
the checking step comprising the step of:
searching a drug file containing information regarding drug to drug interaction problems with the new drug and the drugs of the patient as a search criterion.

38. The method of claim 32, the receiving medical data step for the drug interaction analysis code comprising the step of:
receiving data regarding medical conditions of the patient; and
the checking step comprising the step of:
searching a medical condition file containing information regarding drug interaction problems with the new drug and the medical conditions of the patient as a search criterion.

39. The method of claim 28, the receiving medical data step for the drug interaction analysis code comprising the step of:
receiving data regarding allergies of the patient; and
the checking step comprising the step of:
searching a file containing information regarding the drug interaction problems based on allergies with the new drug and the allergies of the patient as a search criterion.

40. The method of claim 32, the receiving medical data step for the drug interaction analysis code comprising the step of:
receiving data regarding food of the patient; and
the checking step comprising the step of:
searching a file containing information regarding drug interaction problems based on food with the new drug and the food of the patient as a search criterion.

41. A processor readable medium adapted for patient specific drug dosing by a computer system, storing:
patient specific drug dosing code for causing a processor of the computer system to perform the steps of:
receiving medical data for a patient;
receiving an indication of a new drug for the patient;
detecting if the new drug provides a narrow therapeutic index;
receiving a first set of dosing parameters for the patient if the new drug provides a narrow therapeutic index; and
calculating a second set of dosing parameters for the patient from the medical data of the patient and the first set of dosing parameters.

42. The processor readable medium of claim 41, wherein the first set of dosing parameters for the patient comprise a creatinine clearance, a volume of distribution, modifying factors, infusion time, and dosing interval.

43. The processor readable medium of claim 41, wherein the second set of dosing parameters comprise a patient kinetic constant, a half-life, and a dosage of the drug.

44. The processor readable medium of claim 41, the patient specific drug dosing code when executed causing the processor to perform the further steps of:
receiving a desired dose of the drug; and
calculating a peak drug value for the patient and a trough drug value for the patient from the desired dose of the drug, the first set of dosing parameters, the second set of dosing parameters, and the medical data for the patient.

45. The processor readable medium of claim 41, patient specific drug dosing code when executed causing the processor to perform the further steps of:
entering an improved dosing stage;
receiving a third set of dosing parameters for the patient;
calculating a fourth set of dosing parameters for the patient from the medical data for the patient and the third set of dosing parameters.

46. The processor readable medium of claim 45, wherein the third set of dosing parameters comprise a maintenance dose, a creatinine dosing, a peak blood draw, a trough blood draw, an infusion length, time of the peak blood draw, a time of the trough blood draw, and an infusion time.

47. The processor readable medium of claim 45, wherein the fourth set of dosing parameters comprise a volume of distribution.

48. The processor readable medium of claim 41, the patient specific drug dosing code when executed causing the processor to perform the further steps of:
receiving a peak drug value for the patient and a trough drug value for the patient; and
calculating a maintenance dose of the drug and a maintenance interval of the drug from the peak draw of value for the patient and the trough drug value for the patient.

49. The processor readable medium of claim 41, the patient specific drug dosing code when executed causing the processor to perform the further steps of:
receiving a maintenance dose of the drug and a maintenance interval of the drug; and
calculating the peak drug value for the patient and the trough drug value for the patient from the maintenance dose of the drug and the maintenance interval of the drug.

50. A processor readable medium adapted for drug interaction analysis by a computer system, storing:
drug interaction analysis code for causing a processor of the computer system to perform the steps of:
receiving medical data for the patient;
receiving an indication of a new drug for the patient;
checking the new drug for a drug interaction problem;
indicating a drug interaction problem if a drug interaction problem is detected;
selecting an alternative drug for the patient if the alternative drug is provided; and
selecting the new drug for the patient if an override command is received.

51. The processor readable medium of claim 50, the receiving medical data step for the drug interaction analysis code comprising the step of:
receiving data regarding drugs of the patient; and
the checking step comprising the step of:
checking the new drug for a drug interaction problem based on an interaction between the new drug and the drugs of the patient.

52. The processor readable medium of claim 50, the receiving medical data step for the drug interaction analysis code comprising the step of:
receiving data regarding medical conditions of the patient; and
the checking step comprising the step of:
checking the new drug for a drug interaction problem based on an interaction between the new drug and medical conditions of the patient.

53. The processor readable medium of claim 50, the receiving medical data step for the drug interaction analysis code comprising the step of:
receiving data regarding a diet of the patient; and
the checking step comprising the step of:
checking the new drug for a drug interaction problem based on an interaction between the new drug and the diet of the patient.

54. The processor readable medium of claim 50, the receiving medical data step for the drug interaction analysis code comprising the step of:
receiving data regarding allergies of the patient; and
the checking step further comprising the step of:
checking the new drug for a drug interaction problem based on an interaction between the drug and the allergies of the patient.

55. The processor readable medium of claim 50, the receiving medical data step for the drug interaction analysis code comprising the step of:
receiving data regarding drugs of the patient; and
the checking step comprising the step of:
searching a drug file containing information regarding drug to drug interaction problems with the new drug and the drugs of the patient as a search criterion.

56. The processor readable medium of claim 50, the receiving medical data step for the drug interaction analysis code comprising the step of:
receiving data regarding medical conditions of the patient; and
the checking step comprising the step of:
searching a medical condition file containing information regarding drug interaction problems with the new drug and the medical conditions of the patient as a search criterion.

57. The processor readable medium of claim 50, the receiving medical data step for the drug interaction analysis code comprising the step of:
receiving data regarding allergies of the patient; and
the checking step comprising the step of:
searching a file containing information regarding the drug interaction problems based on allergies with the new drug and the allergies of the patient as a search criterion.

58. The processor readable medium of claim 50, the receiving medical data step for the drug interaction analysis code comprising the step of:
receiving data regarding food of the patient; and
the checking step comprising the step of:
searching a file containing information regarding drug interaction problems based on food with the new drug and the food of the patient as a search criterion.

59. A processor readable medium adapted for order generation by a computer system storing:
order generation code for causing a processor of the computer system to perform the steps of:
providing an order on a display screen of the computer system after the drug interaction analysis code is executed;
receiving order information for the new drug into the order; and
processing the order.

60. The processor readable medium of claim 59, storing:
the order generation code when executed causing the processor to perform the further step of:
providing the order to a printer coupled to the computer system.

61. A pharmacy drug management computer system, comprising:
a processor; and
a medium readable by the processor storing drug management code providing access to drug doser for dosing drugs for a patient having a narrow therapeutic index, a drug interaction analyzer for detecting drug interaction problems for a patient, and an order generator for generating an order for a patient.

62. The pharmacy drug management computer system of claim 61, the drug management code providing access to a patient data matching database for locating therapies of patients with medical data matching medical data of a patient.

63. The pharmacy drug management computer system of claim 61, the drug management code providing access to advisories providing information concerning drugs.

64. The pharmacy drug management computer system of claim 61, the drug management code providing access to a formulary listing available drugs.

65. The pharmacy drug management computer system of claim 61, the drug management code providing access to drug information help files.
